(19) United States
(12) Patent Application Publication
(10) Pub. No.: US 2026/0137678 A1
(43) Pub. Date: May 21, 2026

(54) METHODS FOR TREATING OR PREVENTING NEUROENDOCRINE TUMOR FORMATION USING XPO1 INHIBITORS

(71) Applicants: MEMORIAL SLOAN-KETTERING CANCER CENTER, New York, NY (US); MEMORIAL HOSPITAL FOR CANCER AND ALLIED DISEASES, New York, NY (US); SLOAN-KETTERING INSTITUTE FOR CANCER RESEARCH, New York, NY (US)

(72) Inventors: Álvaro D. Quintanal Villalonga, New York, NY (US); Charles Michael Rudin, New York, NY (US)

(73) Assignees: MEMORIAL SLOAN-KETTERING CANCER CENTER, New York, NY (US); MEMORIAL HOSPITAL FOR CANCER AND ALLIED DISEASES, New York, NY (US); SLOAN-KETTERING INSTITUTE FOR CANCER RESEARCH, New York, NY (US)

(21) Appl. No.: 19/122,192

(22) PCT Filed: Oct. 17, 2023
(86) PCT No.: PCT/US2023/077068

§ 371 Date: Apr. 17, 2025

Related U.S. Application Data
(60) Provisional application No. 63/417,158, filed on Oct. 18, 2022.

(51) Int. Cl. A61K 31/497; A61K 31/4166; A61K 31/506; A61K 31/7048; A61K 33/243; A61P 35/00

(52) U.S. Cl. CPC A61K 31/497; A61K 31/4166; A61K 31/506; A61K 31/7048; A61K 33/243; A61P 35/00

(57) ABSTRACT

The present disclosure provides methods for treating or preventing neuroendocrine tumor formation in subjects diagnosed with TP53 and RBI deficient adenocarcinomas (e.g., lung or prostate adenocarcinomas) using XP01 inhibitors. Also disclosed herein are methods for preventing neuroendocrine tumor formation in subjects diagnosed with adenocarcinomas (e.g., TP53 and RBI deficient lung or prostate adenocarcinomas) using XP01 inhibitors in combination with androgen receptor (AR) inhibitors, epidermal growth factor receptor (EGFR) inhibitors, or chemotherapeutic drugs.

### CROSS-REFERENCE TO RELATED APPLICATIONS

This application claims the benefit of and priority to U.S. Provisional Ser. No. 63/417,158 filed Oct. 18, 2022, the entire contents of which are incorporated herein by reference.

### STATEMENT OF GOVERNMENT SUPPORT

This invention was made with government support under CA197936, and U24 CA213274, CA264078-01 and CA08748, awarded by the National Institutes of Health. The government has certain rights in the invention.

### TECHNICAL FIELD

The present disclosure provides methods for treating or preventing neuroendocrine tumor formation in subjects diagnosed with TP53 and RB1 deficient adenocarcinomas (e.g., lung or prostate adenocarcinomas) using XPO1 inhibitors. Also disclosed herein are methods for preventing neuroendocrine tumor formation in subjects diagnosed with adenocarcinomas (e.g., TP53 and RB1 deficient lung or prostate adenocarcinomas) using XPO1 inhibitors in combination with androgen receptor (AR) inhibitors, epidermal growth factor receptor (EGFR) inhibitors, or chemotherapeutic drugs.

### BACKGROUND

The following description of the background of the present technology is provided simply as an aid in understanding the present technology and is not admitted to describe or constitute prior art to the present technology.

Lineage plasticity, the capacity of cells to transition from one committed identity to that of a distinct developmental lineage, can promote survival of cancer cells under unfavorable conditions such as oncogenic driver-targeted therapy. Under the selective pressure of targeted therapies, histological transformation of adenocarcinoma (AD) to highly aggressive neuroendocrine (NE) derivatives resembling small cell carcinomas has been reported in up to 20% of AR-dependent prostate cancers and up to 14% of EGFR-mutant lung ADs. It has been described that tumors with both TP53 and RB1 mutations/loss show increased susceptibility to transformation, thus defining a patient population at risk. NE transformation in both disease contexts is associated with notably poor prognoses. Little is known about the molecular alterations driving NE transformation in human tumors, in part due to the absence of viable models to study this phenomenon, and in part to the paucity of transformation samples available for molecular analysis. To date (1) no specific therapies for NE transformation prevention are available for patients at high risk of transformation, and (2) the primary therapy available for NE-transformed patients, platinum doublet (cisplatin and etoposide, plus immunotherapy in the lung cancer setting), show only short-term responses.

Accordingly, there is an urgent need for effective therapies for treating or preventing NE transformation in adenocarcinoma patients.

### SUMMARY OF THE PRESENT TECHNOLOGY

In one aspect, the present disclosure provides a method for treating or preventing neuroendocrine tumor formation in a patient diagnosed with adenocarcinoma comprising administering to the patient an effective amount of an XPO1 inhibitor, wherein the adenocarcinoma exhibits reduced expression and/or activity of TP53 and RB1. In some embodiments, the adenocarcinoma comprises (a) a genetic mutation, an indel, a copy number alteration or epigenetic downregulation in TP53 and (b) a genetic mutation, an indel, a copy number alteration or epigenetic downregulation in RB1.

In another aspect, the present disclosure provides a method for preventing neuroendocrine tumor formation in a patient diagnosed with adenocarcinoma comprising administering to the patient an effective amount of an XPO1 inhibitor and an effective amount of at least one chemotherapeutic drug, wherein the adenocarcinoma exhibits reduced expression and/or activity of TP53 and RB1. Also provided herein is a method for enhancing responsiveness of a patient with neuroendocrine tumors to systemic chemotherapy comprising administering to the patient an effective amount of an XPO1 inhibitor and an effective amount of at least one chemotherapeutic drug, wherein the neuroendocrine tumors exhibit reduced expression and/or activity of TP53 and RB1. In some embodiments, the adenocarcinoma or the neuroendocrine tumors comprise(s) (a) a genetic mutation, an indel, a copy number alteration or epigenetic downregulation in TP53 and (b) a genetic mutation, an indel, a copy number alteration or epigenetic downregulation in RB1. In some embodiments, the XPO1 inhibitor and the at least one chemotherapeutic drug are administered sequentially, simultaneously, or separately. Additionally or alternatively, in some embodiments, the at least one chemotherapeutic drug is administered orally, intranasally, parenterally, intravenously, intramuscularly, intraperitoneally, or subcutaneously, intratumorally, or topically. The at least one chemotherapeutic drug may be an alkylating agent, a platinum agent, a taxane, a vinca agent, an aromatase inhibitor, a cytostatic alkaloid, a cytotoxic antibiotic, an antimetabolite, an endocrine/hormonal agent, or a bisphosphonate therapy agent. Examples of chemotherapeutic drugs include, but are not limited to cyclophosphamide, fluorouracil (or 5-fluorouracil or 5-FU), methotrexate, edatrexate (10-ethyl-10-deaza-aminopterin), thiotepa, carboplatin, cisplatin, taxanes, paclitaxel, protein-bound paclitaxel, docetaxel, vinorelbine, tamoxifen, raloxifene, toremifene, fulvestrant, gemcitabine, irinotecan, ixabepilone, temozolmide, topotecan, vincristine, vinblastine, eribulin, mutamycin, capecitabine, anastrozole, exemestane, letrozole, risedronate, pamidronate, ibandronate, alendronate, denosumab, zoledronate, tykerb, anthracyclines (e.g., daunorubicin and doxorubicin), oxaliplatin, melphalan, etoposide, mechlorethamine, bleomycin, microtubule poisons, annonaceous acetogenins, abraxane, leucovorin, nab-paclitaxel, everolimus, pegylated-hyaluronidase, pemetrexed, folinic acid, MK2206, GDC-0449, IPI-926, M402, LY293111 or combinations thereof.

In yet another aspect, the present disclosure provides a method for preventing neuroendocrine tumor formation in a patient diagnosed with adenocarcinoma comprising administering to the patient an effective amount of an XPO1 inhibitor and an effective amount of an androgen receptor (AR) inhibitor. The adenocarcinoma may exhibit reduced expression and/or activity of TP53 and RB1. In certain embodiments, the adenocarcinoma comprises (a) a genetic mutation, an indel, a copy number alteration or epigenetic downregulation in TP53 and (b) a genetic mutation, an indel, a copy number alteration or epigenetic downregulation in RB1. Additionally or alternatively, in some embodiments, the XPO1 inhibitor and the AR inhibitor are administered sequentially, simultaneously, or separately. Additionally or alternatively, in some embodiments, the AR inhibitor is administered orally, intranasally, parenterally, intravenously, intramuscularly, intraperitoneally, or subcutaneously, intratumorally, or topically. Examples of AR inhibitors include, but are not limited to apalutamide, bicalutamide, darolutamide, enzalutamide, flutamide, abiraterone acetate, ARN-509, and nilutamide.

In another aspect, the present disclosure provides a method for preventing neuroendocrine tumor formation in a patient diagnosed with adenocarcinoma comprising administering to the patient an effective amount of an XPO1 inhibitor and an effective amount of an epidermal growth factor receptor (EGFR) inhibitor (EGFRi) (e.g., EGFR tyrosine kinase inhibitor (TKI), anti-EGFR antibodies). The adenocarcinoma may exhibit reduced expression and/or activity of TP53 and RB1. In certain embodiments, the adenocarcinoma comprises (a) a genetic mutation, an indel, a copy number alteration or epigenetic downregulation in TP53 and (b) a genetic mutation, an indel, a copy number alteration or epigenetic downregulation in RB1. Additionally or alternatively, in some embodiments, the XPO1 inhibitor and the EGFRi are administered sequentially, simultaneously, or separately. Additionally or alternatively, in some embodiments, the EGFRi is administered orally, intranasally, parenterally, intravenously, intramuscularly, intraperitoneally, or subcutaneously, intratumorally, or topically. Examples of EGFRis include, but are not limited to, osimertinib, afatinib, erlotinib, gefitinib, icotinib, dacomitinib, rociletinib, olmutinib, cetuximab, panitumumab, nimotuzumab, and necitumumab.

In any and all embodiments of the methods disclosed herein, the patient is diagnosed with TP53^{-/-} and RB1^{-/-} mutant adenocarcinoma. In some embodiments, the TP53^{-/-} and RB1^{-/-} mutant adenocarcinoma is lung adenocarcinoma or prostate adenocarcinoma. Additionally or alternatively, in some embodiments, the XPO1 inhibitor is administered orally, intranasally, parenterally, intravenously, intramuscularly, intraperitoneally, or subcutaneously, intratumorally, or topically. Examples of XPO1 inhibitors include, but are not limited to KPT-330 (selinexor), KOS-2462, KPT-335 (verdinexor), KPT-176, KPT-127, KPT-185, KPT-276, KPT-251, KPT-205, KPT-227, Leptomycin B (LMB), ratjadone, goniothalamin, N-azolylacrylates, anguinomycin, and CBS9106.

In any and all embodiments of the methods disclosed herein, the patient is human. Additionally or alternatively, in some embodiments, the patient is non-responsive to at least one prior line of cancer therapy such as chemotherapy.

### BRIEF DESCRIPTION OF THE DRAWINGS

FIGS. 1A-1H: Exportin 1 is upregulated during NE transformation in the lung and prostate and Exportin 1 inhibition sensitizes NE-transformed lung and prostate tumors to chemotherapy. Exportin 1 mRNA expression (FIG. 1A) and protein abundance (FIG. 1B) in lung tumor clinical specimens, categorized as control never transformed adenocarcinomas (LUAD, RNA n=11, protein n=46), transforming adenocarcinomas (T-LUAD, RNA n=11, protein n=10) and small cell carcinomas (T-SCLC, RNA n=11, protein n=20) and control de novo small cell carcinomas (SCLC, RNA n=16, protein n=32). For (FIG. 1B), H-score medians and standard deviation (top) and representative IHC images (bottom) are shown. FIG. 1C: Exportin 1 mRNA expression in PRAD tumors with (n=22) or without (n=210) NE features. Data from Abida et al., PNAS 2019)¹⁵. FIG. 1D: Exportin 1 mRNA expression in PRADs (n=8), large cell NE carcinomas (LCNEC) and small cell carcinomas (SCPC) (n=9) of the prostate. Data from Tzelepi et al., CCR 2021)¹⁴. FIG. 1E: Exportin 1 protein expression in PRAD (n=21) and NEPC (n=15) clinical specimens, as assessed by IHC. H-score medians and standard deviation (right) and representative images (left) are shown. p-value legend: *<0.05, **<0.01, ***<0.001. FIG. 1F: In vitro synergy assays in Lx1042 (T-SCLC) and H660 (NEPC) cell lines of the combination of selinexor and cisplatin with average synergy score displayed, as assessed by Zero Interaction Potency (ZIP) and calculated using the SynergyFinder web application (2.0). Representative plot is shown. FIG. 1G: In vivo treatments of Lx1042 (T-SCLC) and LuCAP49 (NEPC) PDXs to compare the efficacy of the combination of cisplatin and selinexor versus that of cisplatin and etoposide. 4-8 female 6-week-old NOD. Cg-Prkdc<scid>Il2rg<tm1Wjl>/SzJ (NSG) mice (PDXs) were subcutaneously engrafted per treatment arm and until tumors reached 100-150 mm³. At that point, mice were randomized into groups and treated with either vehicle (n=8), cisplatin (2 mg/kg intraperitoneally (i.p.) once/week, n=4 for Lx1042 and n=5 for LuCAP49), etoposide (3 mg/kg i.p. QDx3, n=4 for Lx1042 and n=5 for LuCAP49), selinexor (10 mg/kg per os (p.o.) QDx3, n=4 for Lx1042 and n=5 for LuCAP49), or the combinations of cisplatin+etoposide (n=8) or cisplatin+selinexor (n=8). Mice weights and tumor volumes were measured twice a week and mice were sacrificed when tumors reached humane endpoint (volume=1000 mm³). p-values were calculated using the Student's t-test (unpaired, heterogeneous variances, two-tailed). FIG. 1H: Representative western blots showing the activation of the AKT/mTOR pathway in tumors derived from FIG. 1G. p-value legend: *<0.05, **<0.01, *<0.001.

FIGS. 2A-2E. Loss of TP53/RB1 function induces exportin 1 expression and sensitivity to selinexor. FIG. 2A: XPO1 mRNA expression in LUAD clinical specimens, categorized by their TP53/RB1 status. Data obtained from LUAD TCGA (PanCancer, n=237 wild type (wt), 33 mutated), LUAD OncoSG (OncoSG, Nat Genetics 2020, n=109 wt, 6 mutated) and PRAD TCGA (PanCancer, n=367 wt, 6 mutated)^{14,15}. FIG. 2B: Western blot showing exportin 1 protein abundance in isogenic H1563 (LUAD) and 22PC (PRAD) cell lines with or without induced loss of function of TP53 and/or RB1 and western blot quantification (n=2) (see methods). FIG. 2C: Barplot showing a representative biological replicate of an experiment assessing viability of control and TP53/RB1-loss (DKO) H1563, 22PC and LnCap (PRAD) cells treated with 5 nM selinexor. Each of the conditions shown was normalized to their respective untreated condition, and represented as a normalized viability percentage. For FIGS. 2B-2C, p-values were calculated using the Student's t-test (unpaired, heterogeneous variances, two-tailed). p-value legend: *<0.05, **<0.01, ***<0.001. FIG. 2D: XPO1 mRNA expression in isogenic H1563 (LUAD) and 22PC (PRAD) cell lines with or without induced loss of function of TP53 and/or RB1 by shRNA against RB1 and dominant negative TP53 gene overexpression (H1563) or CRISPR/Cas9 knock out (22PC). FIG. 2E: Barplot exhibiting data from XPO1 gene promoter reporter assays in isogenic H1563 (LUAD) and 22PC (PRAD) cell lines with or without induced loss of function of TP53 and/or and RB, or with E2F1 overexpression. Normalized luciferase activity of a representative biological replicate is shown.

FIG. 3A: XPO1 mRNA expression in adenocarcinoma clinical specimens, categorized by their TP53/RB1 status. Data obtained from LUAD TCGA (PanCancer), LUAD OncoSG (OncoSG, Nat Genetics 2020) and PRAD TCGA (PanCancer). FIG. 3B: Full blot images from FIG. 2B. FIG. 3C: DNA accessibility ATACseq data from isogenic control and TP 53/RB1-inactivated H1563 and 22PC isogenic cell lines. The transcription start site for the XPO1 gene is highlighted. FIG. 3D: Binding score for TP53 and E2F1 in the transcription start site of the XPO1 gene in different experimental settings including specimens from lung, prostate and other sites. Data obtained from The Signaling Pathways Project (ChIP-seq Atlas). FIG. 3E: Plot showing a representative biological replicate of an experiment assessing viability of control and TP 53- and/or RB1-inactivated H1563 and 22PC cells treated with 5 nM selinexor.

FIGS. 4A-4I: Exportin 1 inhibition interferes with NE transformation. In vivo treatment of cell line xenografts for TP53/RB1 DKO 22PC (FIG. 4A) and LnCap/AR (FIG. 4D) PRAD cells with enzalutamide, selinexor or their combination. 5-10 female (22PC) or male (LnCap/AR) 6-week-old athymic nude mice were subcutaneously engrafted per treatment arm and until tumors reached 100-150 mm³. At that point, mice were randomized into groups and treated with either vehicle (n=7 for 22PC and n=4 for LnCap/AR), selinexor (10 mg/kg p.o. QDx3, n=7 for 22PC and n=4 for LnCap/AR), enzalutamide (10 mg/kg p.o. QDx5, n=7 for 22PC and n=5 for LnCap/AR), or the combinations of enzalutamide+selinexor at the previously mentioned doses (n=9 for 22PC and n=5 for LnCap/AR). Mice weights and tumor volumes were measured twice a week and mice were sacrificed when tumors reached humane endpoint (volume=1000 mm³). Tumor volumes are shown as normalized volume in arbitrary units (au). Each tumor was normalized to its volume at day 0 of treatment. Representative IHC images for synaptophysin (SYP) and chromogranin A (CHGA) staining in DKO 22PC (FIG. 4B) and LnCap/AR (FIG. 4E) tumors. Quantification of SYP- or CHGA-positive cells, normalized to tissue area, in immunohistochemical tissue stains in DKO 22PC (n=6, 5, 4 and 4 tumor for control, selinexor-, enzalutamide and combo-treated arms, respectively) (FIG. 4C) and LnCap/AR (FIG. 4F) (n=5, 5, 6 and 6 randomly selected tissue pieces for control, selinexor-, enzalutamide and combo-treated arms, respectively) tumors. Positive cells were counted, tissue area (viable tumor area) was estimated using the SketchAndCalc online app (https://www.sketchandcalc.com/), and positive-stained cells were normalized by estimated area. FIG. 4G: RNA sequencing data from tumors from FIG. 4A collected at control arm experimental endpoint (day 31), showing mRNA expression for genes of interest, involved in NE transformation, divided by treatment arm (n=4, 3, 3 and 3 tumors for the control, enzalutamide-, selinexor- and combo-treated tumors). mRNA expression values are shown as Transcript per Million (TPM). FIG. 4H: H&E and IHC staining for markers of interest for the EGFR-mutant combined NSCLC/SCLC PDX tumor MSK_Lx151. FIG. 4I: In vivo treatment of the MSK_Lx151 PDX with vehicle (n=5), Osimertinib (n=5), selinexor (n=5) or their combination (n=5). 5-10 female 6-week-old NOD. Cg-Prkdc<scid>Il2rg<tm1Wjl>/SzJ (NSG) mice were subcutaneously engrafted per treatment arm and until tumors reached 100-150 mm³. At that point, mice were randomized into groups and treated with either vehicle, selinexor (10 mg/kg p.o. QDx3), enzalutamide (10 mg/kg per os (p.o). QDx5), osimertinib (25 mg/kg p.o. QDx5) or the combination of osimertinib+selinexor at the previously mentioned doses. Mice weights and tumor volumes were measured twice a week and mice were sacrificed when tumors reached humane endpoint (volume=1000 mm³). Tumor volumes are shown as normalized volume in arbitrary units (au). Each tumor was normalized to its volume at day 0 of treatment. For FIGS. 4A, 4C, 4D and 4F, P-values were calculated using the Student's t-test (unpaired, heterogeneous variances, two-tailed). P-value legend: *<0.05, **<0.01, ***<0.001, ns, not significant.

FIG. 5A: Plot showing mouse body weight for the mice treated with enzalutamide, selinexor or their combination in FIG. 3A. FIG. 5B: Pathway enrichment analysis on DEGs from selinexor-treated versus control untreated de novo SCLC cell lines H69 and H82. FIG. 5C: SOX2 mRNA expression on control and selinexor-treated de novo SCLC cell lines H69 and H82. Data for FIGS. 5B-5C was obtained from¹¹.

FIG. 5D: Selinexor prevented the acquisition of a basal-like phenotype, previously described to be potentiated in TP53/RB1-deficient PRAD after targeted therapy treatment, in parallel with NE features.

FIG. 5E: The combination of Osimertinib and selinexor did not cause additional toxicity compared to osimertinib monotherapy.

FIG. 5F: No significant differences were observed in tumors treated with Osimertinib, Selinexor or both in terms of expression of the LUAD marker TTF-1 and the NE markers synaptophysin and chromogranin A.

FIGS. 6A-6J: Exportin 1 inhibition downregulates SOX2 expression, hindering the acquisition of NE features. Pathway enrichment analysis on DEGs from enzalutamide versus control (FIG. 6A) and combo versus enzalutamide (FIG. 6B) conditions in the transcriptomic data from TP53/RB1 DKO 22PC xenografts treated in vivo and collected at control arm experimental endpoint (day 31). Categorized pathways of interest, previously involved in NE transformation^{5,6}, are shown. FIG. 6C: SOX2 mRNA expression in tumors divided by treatment condition. mRNA expression values are shown as TPM (n=4, 3, 3 and 3 tumors for the control, enzalutamide-, selinexor- and combo-treated tumors). FIG. 6D: Western blot showing SOX2 protein expression of TP53/RB1 DKO 22PC and Lx151 models treated in vivo (n=3 per treatment condition). 5-10 female 6-week-old NOD. Cg-Prkdc<scid>Il2rg<tm1Wjl>/SzJ (NSG) mice (Lx151) or female 6-week-old athymic nude mice (22PC xenografts) were subcutaneously engrafted per treatment arm and until tumors reached 100-150 mm³. At that point, mice were randomized into groups and treated with either vehicle (n=7 for 22PC and n=5 for Lx151) selinexor (10 mg/kg p.o. QDx3, n=7 for 22PC and n=5 for Lx151), enzalutamide (For 22PC, 10 mg/kg per os (p.o), QDx5, n=7), osimertinib (For Lx151, 25 mg/kg p.o. QDx5, n=5) or the combinations of enzalutamide+selinexor (22PC, n=9) or osimertinib+selinexor (Lx151, n=5) at the previously mentioned doses. Mice weights and tumor volumes were measured twice a week and mice were sacrificed when tumors reached humane endpoint (volume=1000 mm³). FIG. 6E: SOX2 mRNA expression in LUAD clinical specimens, categorized by their TP53/RB1 status. p-values are shown. Data obtained from LUAD TCGA (PanCancer, n=237 wt and 33 mutated), LUAD OncoSG (OncoSG, Nat Genetics 2020, n=109 wt and mutated) and PRAD TCGA (PanCancer, n=107 wt and 19mutated)^{14,15}. See also FIG. 7. FIG. 6F: Western blot showing SOX2 protein expression in isogenic control and TP53- and/or RB1-inactivated H1563 (LUAD) and 22PC (PRAD) cell lines. FIG. 6G: SOX2 protein expression in control and TP53/RB1-inactivated H1563 (LUAD) and 22PC and LnCap (PRAD) cell lines treated with selinexor 5 nM for 4 days. FIG. 6H shows the effects of enzalutamide-, selinexor- and combination-treatment in SOX2 overexpressing tumors relative to non-SOX2 overexpressing tumors. SOX2, CHGA, NCAM1, SYP and AR protein abundance (FIG. 6I) and ASCL1, CHGA and INSM1 mRNA expression (FIG. 6J) in DKO 22PC and DKO LnCAP cells treated with enzalutamide (150 nM), selinexor (5 nM) or their combination for 4 days. For western blots and mRNA plots, representative images are shown. p-values were calculated using the Student's t-test (unpaired, heterogeneous variances, two-tailed). p-value legend: *<0.05, **<0.01, ***<0.001.

FIG. 7A: SOX2 mRNA expression in adenocarcinoma clinical specimens, categorized by their TP53/RB1 status. Data obtained from LUAD TCGA (PanCancer), LUAD OncoSG (OncoSG, Nat Genetics 2020) and PRAD TCGA (PanCancer).

FIG. 7B: SOX2 mRNA expression was elevated in isogenic TP53/RB1-inactivated LUAD and PRAD cell lines. This induction of SOX2 was prevented by selinexor treatment.

FIG. 7C: Overexpression of POU3F2, ONECUT2, or a constitutively active isoform of AKT (myrAKT), did not rescue NE phenotype inhibited by Selinexor in TP53/RB1-deficient PRAD models.

FIG. 8A: RB and p53 protein abundance by western blot in TP53- and/or RB1-genetically inactivated LUAD (H1563, overexpression of dominant negative TP53 and short hairpin RNA against RB1) and PRAD (22PC, CRISPR-Cas9 inactivation) isogenic cell lines.

FIG. 8B: E2F1 protein abundance in LUAD (H1563, overexpression of dominant negative TP53 and short hairpin RNA against RB1) and PRAD (22PC, CRISPR-Cas9 inactivation) isogenic cell lines.

FIG. 8C shows increased selinexor sensitivity in LUAD (H1563), and PRAD (22PC and LnCap) cell lines with TP53/RB1 inactivation relative to wild-type controls.

FIGS. 8D-8E: Proliferation assays were performed in TP53/RB1-inactivated 22PC and H 1563 cell lines and the cell cycle profile was assessed in those being treated with selinexor. We observed a slight increase in proliferation after inactivation of TP53 and RB1, with no significant shifts in cell cycle profile even at substantially cytotoxic concentrations of selinexor.

### DETAILED DESCRIPTION

It is to be appreciated that certain aspects, modes, embodiments, variations and features of the present methods are described below in various levels of detail in order to provide a substantial understanding of the present technology.

In practicing the present methods, many conventional techniques in molecular biology, protein biochemistry, cell biology, immunology, microbiology and recombinant DNA are used. See, e.g., Sambrook and Russell eds. (2001) Molecular Cloning: A Laboratory Manual, 3rd edition; the series Ausubel et al. eds. (2007) Current Protocols in Molecular Biology, the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization, Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology. Methods to detect and measure levels of polypeptide gene expression products (i.e., gene translation level) are well-known in the art and include the use of polypeptide detection methods such as antibody detection and quantification techniques. (See also, Strachan & Read, Human Molecular Genetics, Second Edition. (John Wiley and Sons, Inc., NY, 1999)).

The present disclosure demonstrate that (1) exportin-1 inhibition delays NE transformation in lung and prostate adenocarcinoma at high risk of transformation when treated with targeted therapy, and (2) exportin-1 inhibition robustly sensitizes NE-transformed lung and prostate tumors to chemotherapy.

### Definitions

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

As used herein, the term “adenocarcinoma” refers to cancer that forms in the glandular tissue, which lines certain internal organs and makes and releases substances in the body, such as mucus, digestive juices, and other fluids. Most cancers of the breast, lung, esophagus, stomach, colon, rectum, pancreas, prostate, and uterus are adenocarcinomas.

As used herein, the “administration” of an agent or drug to a subject includes any route of introducing or delivering to a subject a compound to perform its intended function. Administration can be carried out by any suitable route, including orally, intranasally, parenterally (intravenously, intramuscularly, intraperitoneally, or subcutaneously), intratumorally, or topically. Administration includes self-administration and the administration by another.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease or condition, a positive control (a compound or composition known to exhibit the desired therapeutic effect) and a negative control (a subject or a sample that does not receive the therapy or receives a placebo) are typically employed.

As used herein, the term “effective amount” refers to a quantity sufficient to achieve a desired therapeutic and/or prophylactic effect, e.g., an amount which results in the prevention of, or a decrease in a disease or condition described herein or one or more signs or symptoms associated with a disease or condition described herein. In the context of therapeutic or prophylactic applications, the amount of a composition administered to the subject will vary depending on the composition, the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compositions may be administered to a subject having one or more signs or symptoms of lung or prostate adenocarcinomas. As used herein, a “therapeutically effective amount” of a composition refers to composition levels in which the physiological effects of a disease or condition are ameliorated or eliminated. A therapeutically effective amount can be given in one or more administrations.

As used herein, “expression” includes one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function.

As used herein, the terms “individual”, “patient”, or “subject” are used interchangeably and refer to an individual organism, a vertebrate, a mammal, or a human. In certain embodiments, the individual, patient or subject is a human.

As used herein, “prevention,” “prevent,” or “preventing” of a disorder or condition refers to one or more compounds that, in a statistical sample, reduces the occurrence of the disorder or condition in the treated sample relative to an untreated control sample, or delays the onset of one or more symptoms of the disorder or condition relative to the untreated control sample.

As used herein, a “sample” or “biological sample” refers to a body fluid or a tissue sample isolated from a subject. In some cases, a biological sample may consist of or comprise whole blood, platelets, red blood cells, white blood cells, plasma, sera, urine, feces, epidermal sample, vaginal sample, skin sample, cheek swab, sperm, amniotic fluid, cultured cells, bone marrow sample, tumor biopsies, aspirate and/or chorionic villi, cultured cells, endothelial cells, synovial fluid, lymphatic fluid, ascites fluid, interstitial or extracellular fluid and the like. The term “sample” may also encompass the fluid in spaces between cells, including gingival crevicular fluid, bone marrow, cerebrospinal fluid (CSF), saliva, mucus, sputum, semen, sweat, urine, or any other bodily fluids. Samples can be obtained from a subject by any means including, but not limited to, venipuncture, excretion, ejaculation, massage, biopsy, needle aspirate, lavage, scraping, surgical incision, or intervention or other means known in the art. A blood sample can be whole blood or any fraction thereof, including blood cells (red blood cells, white blood cells or leukocytes, and platelets), serum and plasma.

As used herein, the term “separate” therapeutic use refers to an administration of at least two active ingredients at the same time or at substantially the same time by different routes.

As used herein, the term “sequential” therapeutic use refers to administration of at least two active ingredients at different times. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, the term “simultaneous” therapeutic use refers to the administration of at least two active ingredients by the same route and at the same time or at substantially the same time.

As used herein, the term “therapeutic agent” is intended to mean a compound that, when present in an effective amount, produces a desired therapeutic effect on a subject in need thereof.

“Treating”, “treat”, or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder, and/or (iv) inhibiting, relieving, or slowing progression of one or more symptoms of the disease or disorder. In some embodiments, treatment means that the symptoms associated with the disease are, e.g., alleviated, reduced, cured, or placed in a state of remission.

It is also to be appreciated that the various modes of treatment or prevention of medical diseases and conditions as described are intended to mean “substantial,” which includes total but also less than total treatment or prevention, and wherein some biologically or medically relevant result is achieved. The treatment may be a continuous prolonged treatment for a chronic disease or a single, or few time administrations for the treatment of an acute condition.

### Therapeutic Methods of the Present Technology

In one aspect, the present disclosure provides a method for treating or preventing neuroendocrine tumor formation in a patient diagnosed with adenocarcinoma comprising administering to the patient an effective amount of an XPO1 inhibitor, wherein the adenocarcinoma exhibits reduced expression and/or activity of TP53 and RB1. In some embodiments, the adenocarcinoma comprises (a) a genetic mutation, an indel, a copy number alteration or epigenetic downregulation in TP53 and (b) a genetic mutation, an indel, a copy number alteration or epigenetic downregulation in RB1.

In another aspect, the present disclosure provides a method for preventing neuroendocrine tumor formation in a patient diagnosed with adenocarcinoma comprising administering to the patient an effective amount of an XPO1 inhibitor and an effective amount of at least one chemotherapeutic drug, wherein the adenocarcinoma exhibits reduced expression and/or activity of TP53 and RB1. Also provided herein is a method for enhancing responsiveness of a patient with neuroendocrine tumors to systemic chemotherapy comprising administering to the patient an effective amount of an XPO1 inhibitor and an effective amount of at least one chemotherapeutic drug, wherein the neuroendocrine tumors exhibit reduced expression and/or activity of TP53 and RB1. In some embodiments, the adenocarcinoma or the neuroendocrine tumors comprise(s) (a) a genetic mutation, an indel, a copy number alteration or epigenetic downregulation in TP53 and (b) a genetic mutation, an indel, a copy number alteration or epigenetic downregulation in RB1. In some embodiments, the XPO1 inhibitor and the at least one chemotherapeutic drug are administered sequentially, simultaneously, or separately. Additionally or alternatively, in some embodiments, the at least one chemotherapeutic drug is administered orally, intranasally, parenterally, intravenously, intramuscularly, intraperitoneally, subcutaneously, intratumorally, topically, by inhalation spray, buccally, or via an implanted reservoir. The at least one chemotherapeutic drug may be an alkylating agent, a platinum agent, a taxane, a vinca agent, an aromatase inhibitor, a cytostatic alkaloid, a cytotoxic antibiotic, an antimetabolite, an endocrine/hormonal agent, or a bisphosphonate therapy agent. Examples of chemotherapeutic drugs include, but are not limited to cyclophosphamide, fluorouracil (or 5-fluorouracil or 5-FU), methotrexate, edatrexate (10-ethyl-10-deaza-aminopterin), thiotepa, carboplatin, cisplatin, taxanes, paclitaxel, protein-bound paclitaxel, docetaxel, vinorelbine, tamoxifen, raloxifene, toremifene, fulvestrant, gemcitabine, irinotecan, ixabepilone, temozolmide, topotecan, vincristine, vinblastine, eribulin, mutamycin, capecitabine, anastrozole, exemestane, letrozole, risedronate, pamidronate, ibandronate, alendronate, denosumab, zoledronate, tykerb, anthracyclines (e.g., daunorubicin and doxorubicin), oxaliplatin, melphalan, etoposide, mechlorethamine, bleomycin, microtubule poisons, annonaceous acetogenins, abraxane, leucovorin, nab-paclitaxel, everolimus, pegylated-hyaluronidase, pemetrexed, folinic acid, MK2206, GDC-0449, IPI-926, M402, LY293111 or combinations thereof.

In yet another aspect, the present disclosure provides a method for preventing neuroendocrine tumor formation in a patient diagnosed with adenocarcinoma comprising administering to the patient an effective amount of an XPO1 inhibitor and an effective amount of an androgen receptor (AR) inhibitor. The adenocarcinoma may exhibit reduced expression and/or activity of TP53 and RB1. In certain embodiments, the adenocarcinoma comprises (a) a genetic mutation, an indel, a copy number alteration or epigenetic downregulation in TP53 and (b) a genetic mutation, an indel, a copy number alteration or epigenetic downregulation in RB1. Additionally or alternatively, in some embodiments, the XPO1 inhibitor and the AR inhibitor are administered sequentially, simultaneously, or separately. Additionally or alternatively, in some embodiments, the AR inhibitor is administered orally, intranasally, parenterally, intravenously, intramuscularly, intraperitoneally, subcutaneously, intratumorally, topically, by inhalation spray, buccally, or via an implanted reservoir. Examples of AR inhibitors include, but are not limited to apalutamide, bicalutamide, darolutamide, enzalutamide, flutamide, abiraterone acetate, ARN-509, and nilutamide.

In another aspect, the present disclosure provides a method for preventing neuroendocrine tumor formation in a patient diagnosed with adenocarcinoma comprising administering to the patient an effective amount of an XPO1 inhibitor and an effective amount of an epidermal growth factor receptor (EGFR) inhibitor (EGFRi) (e.g., EGFR tyrosine kinase inhibitor (TKI), anti-EGFR antibodies). The adenocarcinoma may exhibit reduced expression and/or activity of TP53 and RB1. In certain embodiments, the adenocarcinoma comprises (a) a genetic mutation, an indel, a copy number alteration or epigenetic downregulation in TP53 and (b) a genetic mutation, an indel, a copy number alteration or epigenetic downregulation in RB1. Additionally or alternatively, in some embodiments, the XPO1 inhibitor and the EGFRi are administered sequentially, simultaneously, or separately. Additionally or alternatively, in some embodiments, the EGFRi is administered orally, intranasally, parenterally, intravenously, intramuscularly, intraperitoneally, subcutaneously, intratumorally, topically, by inhalation spray, buccally, or via an implanted reservoir. Examples of EGFRis include, but are not limited to, osimertinib, afatinib, erlotinib, gefitinib, icotinib, dacomitinib, rociletinib, olmutinib, cetuximab, panitumumab, nimotuzumab, and necitumumab.

In any and all embodiments of the methods disclosed herein, the patient is diagnosed with TP53^{-/-} and RB1^{-/-} mutant adenocarcinoma. In some embodiments, the TP53^{-/-} and RB1^{-/-} mutant adenocarcinoma is lung adenocarcinoma or prostate adenocarcinoma. Additionally or alternatively, in some embodiments, the XPO1 inhibitor is administered orally, intranasally, parenterally, intravenously, intramuscularly, intraperitoneally, subcutaneously, intratumorally, topically, by inhalation spray, buccally, or via an implanted reservoir.

Examples of XPO1 inhibitors include, but are not limited to KPT-330 (selinexor), KOS-2462, KPT-335 (verdinexor), KPT-176, KPT-127, KPT-185, KPT-276, KPT-251, KPT-205, KPT-227, Leptomycin B (LMB), ratjadone, goniothalamin, N-azolylacrylates, anguinomycin, and CBS9106.

In any and all embodiments of the methods disclosed herein, the patient is human. Additionally or alternatively, in some embodiments, the patient is non-responsive to at least one prior line of cancer therapy such as chemotherapy.

Additionally or alternatively, in some embodiments of the methods disclosed herein, the XPO1 inhibitor can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), simultaneously with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of the AR inhibitor, EGFRi, or chemotherapeutic drug to the patient.

In some embodiments, the XPO1 inhibitor and the AR inhibitor or EGFRi or chemotherapeutic drug are administered to a patient, for example, a mammal, such as a human, in a sequence and within a time interval such that the inhibitor or drug that is administered first acts together with the inhibitor or drug that is administered second to provide greater benefit than if each inhibitor were administered alone.

For example, the XPO1 inhibitor and the AR inhibitor or EGFRi or chemotherapeutic drug can be administered at the same time or sequentially in any order at different points in time; however, if not administered at the same time, the XPO1 inhibitor and the AR inhibitor or EGFRi or chemotherapeutic drug are administered sufficiently close in time so as to provide the desired therapeutic or prophylactic effect of the combination of the two inhibitors. In one embodiment, the XPO1 inhibitor and the AR inhibitor or EGFRi or chemotherapeutic drug exert their effects at times which overlap. In some embodiments, the XPO1 inhibitor and the AR inhibitor or EGFRi or chemotherapeutic drug are each administered as separate dosage forms, in any appropriate form and by any suitable route. In other embodiments, the XPO1 inhibitor and the AR inhibitor or EGFRi or chemotherapeutic drug are administered simultaneously in a single dosage form.

It will be appreciated that the frequency with which any of these therapeutic agents can be administered once or more than once over a period of about 2 days, about 3 days, about 4 days, about 5 days, about 6 days, about 7 days, about 8 days, about 9 days, about 10 days, about 11 days, about 12 days, about 13 days, about 14 days, about 20 days, about 28 days, about a week, about 2 weeks, about 3 weeks, about 4 weeks, about a month, about every 2 months, about every 3 months, about every 4 months, about every 5 months, about every 6 months, about every 7 months, about every 8 months, about every 9 months, about every 10 months, about every 11 months, about every year, about every 2 years, about every 3 years, about every 4 years, or about every 5 years.

For example, the XPO1 inhibitor or the AR inhibitor or EGFRi or chemotherapeutic drug may be administered daily, weekly, biweekly, or monthly for a particular period of time. The XPO1 inhibitor or the AR inhibitor or EGFRi or chemotherapeutic drug may be dosed daily over a 14 day time period, or twice daily over a seven day time period. The XPO1 inhibitor or AR inhibitor or EGFRi or chemotherapeutic drug may be administered daily for 7 days.

Alternatively, an XPO1 inhibitor or AR inhibitor or EGFRi or chemotherapeutic drug may be administered daily, weekly, biweekly, or monthly for a particular period of time followed by a particular period of non-treatment. In some embodiments, the XPO1 inhibitor or AR inhibitor or EGFRi or chemotherapeutic drug can be administered daily for 14 days followed by seven days of non-treatment, and repeated for two more cycles of daily administration for 14 days followed by seven days of non-treatment. In some embodiments, the XPO1 inhibitor or AR inhibitor or EGFRi or chemotherapeutic drug can be administered twice daily for seven days followed by 14 days of non-treatment, which may be repeated for one or two more cycles of twice daily administration for seven days followed by 14 days of non-treatment.

In some embodiments, the XPO1 inhibitor or AR inhibitor or EGFRi or chemotherapeutic drug is administered daily over a period of 14 days. In another embodiment, the XPO1 inhibitor or AR inhibitor or EGFRi or chemotherapeutic drug is administered daily over a period of 12 days, or 11 days, or 10 days, or nine days, or eight days. In another embodiment, the XPO1 inhibitor or AR inhibitor or EGFRi or chemotherapeutic drug is administered daily over a period of seven days. In another embodiment, the XPO1 inhibitor or AR inhibitor or EGFRi or chemotherapeutic drug is administered daily over a period of six days, or five days, or four days, or three days.

In some embodiments, individual doses of the XPO1 inhibitor and the AR inhibitor or EGFRi or chemotherapeutic drug are administered within a time interval such that the two therapeutic agents can work together (e.g., within 1 hour, 2 hours, 4 hours, 6 hours, 12 hours, 24 hours, 48 hours, 72 hours, 96 hours, 5 days, 6 days, 1 week, or 2 weeks). In some embodiments, the treatment period during which the therapeutic agents are administered is then followed by a non-treatment period of a particular time duration, during which the therapeutic agents are not administered to the patient. This non-treatment period can then be followed by a series of subsequent treatment and non-treatment periods of the same or different frequencies for the same or different lengths of time. In some embodiments, the treatment and non-treatment periods are alternated. It will be understood that the period of treatment in cycling therapy may continue until the patient has achieved a complete response or a partial response, at which point the treatment may be stopped.

Alternatively, the period of treatment in cycling therapy may continue until the patient has achieved a complete response or a partial response, at which point the period of treatment may continue for a particular number of cycles. In some embodiments, the length of the period of treatment may be a particular number of cycles, regardless of patient response. In some other embodiments, the length of the period of treatment may continue until the patient relapses.

In some embodiments, the XPO1 inhibitor and the AR inhibitor or EGFRi or chemotherapeutic drug are each administered at a dose and schedule typically used for that agent during monotherapy. In other embodiments, when the XPO1 inhibitor and the AR inhibitor or EGFRi or chemotherapeutic drug are administered concomitantly, one or both of the agents can advantageously be administered at a lower dose than typically administered when the agent is used during monotherapy, such that the dose falls below the threshold that an adverse side effect is elicited.

The therapeutically effective amounts or suitable dosages of the XPO1 inhibitor and the AR inhibitor or EGFRi or chemotherapeutic drug in combination depends upon a number of factors, including the nature of the severity of the condition to be treated, the particular inhibitor, the route of administration and the age, weight, general health, and response of the individual patient. In certain embodiments, the suitable dose level is one that achieves a therapeutic response as measured by tumor regression or other standard measures of disease progression, progression free survival, or overall survival. In other embodiments, the suitable dose level is one that achieves this therapeutic response and also minimizes any side effects associated with the administration of the therapeutic agent.

Suitable daily dosages of AR inhibitors can generally range, in single or divided or multiple doses, from about 10% to about 120% of the maximum tolerated dose as a single agent. In certain embodiments, the suitable dosages of AR inhibitors are from about 20% to about 100% of the maximum tolerated dose as a single agent. In other embodiments, the suitable dosages of AR inhibitors are from about 25% to about 90% of the maximum tolerated dose as a single agent. In some embodiments, the suitable dosages of AR inhibitors are from about 30% to about 80% of the maximum tolerated dose as a single agent. In other embodiments, the suitable dosages of AR inhibitors are from about 40% to about 75% of the maximum tolerated dose as a single agent. In some embodiments, the suitable dosages of AR inhibitors are from about 45% to about 60% of the maximum tolerated dose as a single agent. In other embodiments, suitable dosages of AR inhibitors are about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 105%, about 110%, about 115%, or about 120% of the maximum tolerated dose as a single agent.

Suitable daily dosages of EGFRis can generally range, in single or divided or multiple doses, from about 10% to about 120% of the maximum tolerated dose as a single agent. In certain embodiments, the suitable dosages of EGFRis are from about 20% to about 100% of the maximum tolerated dose as a single agent. In other embodiments, the suitable dosages of EGFRis are from about 25% to about 90% of the maximum tolerated dose as a single agent. In some embodiments, the suitable dosages of EGFRis are from about 30% to about 80% of the maximum tolerated dose as a single agent. In other embodiments, the suitable dosages of EGFRis are from about 40% to about 75% of the maximum tolerated dose as a single agent. In some embodiments, the suitable dosages of EGFRis are from about 45% to about 60% of the maximum tolerated dose as a single agent. In other embodiments, suitable dosages of EGFRis are about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 105%, about 110%, about 115%, or about 120% of the maximum tolerated dose as a single agent.

Suitable daily dosages of chemotherapeutic drugs can generally range, in single or divided or multiple doses, from about 10% to about 120% of the maximum tolerated dose as a single agent. In certain embodiments, the suitable dosages of chemotherapeutic drugs are from about 20% to about 100% of the maximum tolerated dose as a single agent. In other embodiments, the suitable dosages of chemotherapeutic drugs are from about 25% to about 90% of the maximum tolerated dose as a single agent. In some embodiments, the suitable dosages of chemotherapeutic drugs are from about 30% to about 80% of the maximum tolerated dose as a single agent. In other embodiments, the suitable dosages of chemotherapeutic drugs are from about 40% to about 75% of the maximum tolerated dose as a single agent. In some embodiments, the suitable dosages of chemotherapeutic drugs are from about 45% to about 60% of the maximum tolerated dose as a single agent. In other embodiments, suitable dosages of chemotherapeutic drugs are about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 105%, about 110%, about 115%, or about 120% of the maximum tolerated dose as a single agent.

Suitable daily dosages of XPO1 inhibitors can generally range, in single or divided or multiple doses, from about 10% to about 120% of the maximum tolerated dose as a single agent. In certain embodiments, the suitable dosages of XPO1 inhibitors are from about 20% to about 100% of the maximum tolerated dose as a single agent. In some other embodiments, the suitable dosages of XPO1 inhibitors are from about 25% to about 90% of the maximum tolerated dose as a single agent. In some other embodiments, the suitable dosages of XPO1 inhibitors are from about 30% to about 80% of the maximum tolerated dose as a single agent. In some other embodiments, the suitable dosages of XPO1 inhibitors are from about 40% to about 75% of the maximum tolerated dose as a single agent. In some other embodiments, the suitable dosages of XPO1 inhibitors are from about 45% to about 60% of the maximum tolerated dose as a single agent. In other embodiments, suitable dosages of XPO1 inhibitors are about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 105%, about 110%, about 115%, or about 120% of the maximum tolerated dose as a single agent.

Dosage, toxicity and therapeutic efficacy of any therapeutic agent can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are advantageous. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds may be within a range of circulating concentrations that include the ED50 with little or no toxicity. The dosage may vary within this range depending upon the dosage form employed and the route of administration utilized. For any compound used in the methods, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to determine useful doses in humans accurately. Levels in plasma may be measured, for example, by high performance liquid chromatography.

Typically, an effective amount of the XPO1 inhibitor or AR inhibitor or EGFRi or chemotherapeutic drug, sufficient for achieving a therapeutic or prophylactic effect, may range from about 0.000001 mg per kilogram body weight per day to about 10,000 mg per kilogram body weight per day. Suitably, the dosage ranges are from about 0.0001 mg per kilogram body weight per day to about 100 mg per kilogram body weight per day. For example dosages can be 1 mg/kg body weight or 10 mg/kg body weight every day, every two days or every three days or within the range of 1-10 mg/kg every week, every two weeks or every three weeks. In one embodiment, a single dosage of XPO1 inhibitor or AR inhibitor or EGFRi or chemotherapeutic drug ranges from 0.001-10,000 micrograms per kg body weight. In one embodiment, XPO1 inhibitor or AR inhibitor or EGFRi or chemotherapeutic drug concentrations in a carrier range from 0.2 to 2000 micrograms per delivered milliliter. An exemplary treatment regime entails administration once per day or once a week. In therapeutic applications, a relatively high dosage at relatively short intervals is sometimes required until progression of the disease is reduced or terminated, or until the subject shows partial or complete amelioration of symptoms of disease. Thereafter, the patient can be administered a prophylactic regime.

In some embodiments, a therapeutically effective amount of an XPO1 inhibitor or AR inhibitor or EGFRi or chemotherapeutic drug may be defined as a concentration of the XPO1 inhibitor or AR inhibitor or EGFRi or chemotherapeutic drug at the target tissue of 10⁻¹² to 10⁻⁶ molar, e.g., approximately 10⁻⁷ molar. This concentration may be delivered by systemic doses of 0.001 to 100 mg/kg or equivalent dose by body surface area. The schedule of doses would be optimized to maintain the therapeutic concentration at the target tissue, such as by single daily or weekly administration, but also including continuous administration (e.g., parenteral infusion or transdermal application).

The skilled artisan will appreciate that certain factors may influence the dosage and timing required to effectively treat a subject, including but not limited to, the severity of the disease or disorder, previous treatments, the general health and/or age of the subject, and other diseases present. Moreover, treatment of a subject with a therapeutically effective amount of the therapeutic compositions described herein can include a single treatment or a series of treatments.

The mammal treated in accordance with the present methods can be any mammal, including, for example, farm animals, such as sheep, pigs, cows, and horses; pet animals, such as dogs and cats; laboratory animals, such as rats, mice and rabbits. In some embodiments, the mammal is a human.

### Formulations Including the XPO1 Inhibitors of the Present Technology

The pharmaceutical compositions of the present technology can be manufactured by methods well known in the art such as conventional granulating, mixing, dissolving, encapsulating, lyophilizing, or emulsifying processes, among others. Compositions may be produced in various forms, including granules, precipitates, or particulates, powders, including freeze dried, rotary dried or spray dried powders, amorphous powders, tablets, capsules, syrup, suppositories, injections, emulsions, elixirs, suspensions or solutions. Formulations may optionally contain solvents, diluents, and other liquid vehicles, dispersion or suspension aids, surface active agents, pH modifiers, isotonic agents, thickening or emulsifying agents, stabilizers and preservatives, solid binders, lubricants and the like, as suited to the particular dosage form desired. In certain embodiments, the compositions disclosed herein are formulated for administration to a mammal, such as a human. Formulations including any XPO1 inhibitor disclosed herein may be designed to be short-acting, fast-releasing, or long-acting. In other embodiments, compounds can be administered in a local rather than systemic means, such as administration (e.g., by injection) at a tumor site.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, cyclodextrins, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono-or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. Compositions formulated for parenteral administration may be injected by bolus injection or by timed push, or may be administered by continuous infusion.

In order to prolong the effect of a compound of the present disclosure, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents such as phosphates or carbonates.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings, release controlling coatings and other coatings well known in the pharmaceutical formulating art. They may optionally contain opacifying agents and can also be of a composition that they release the active ingredient(s) only, or in a certain part of the intestinal tract, optionally, in a delayed manner. Examples of embedding compositions that can be used include polymeric substances and waxes.

The active compounds can also be in micro-encapsulated form with one or more excipients as noted above. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or in a certain part of the intestinal tract, optionally, in a delayed manner. Examples of embedding compositions that can be used include polymeric substances and waxes.

### Kits

The present disclosure provides kits comprising one or more XPO1 inhibitors disclosed herein, and instructions for treating or preventing neuroendocrine tumor formation. When simultaneous administration is contemplated, the kit may comprise an XPO1 inhibitor and an AR inhibitor or EGFRi or chemotherapeutic drug that has been formulated into a single pharmaceutical composition such as a tablet, or as separate pharmaceutical compositions. When the XPO1 inhibitor and the AR inhibitor or EGFRi or chemotherapeutic drug are not administered simultaneously, the kit may comprise an XPO1 inhibitor and an AR inhibitor or EGFRi or chemotherapeutic drug that has been formulated as separate pharmaceutical compositions either in a single package, or in separate packages.

Additionally or alternatively, in some embodiments, the kits further comprise at least one AR inhibitor that are useful for treating or preventing neuroendocrine tumor formation. Examples of AR inhibitors include, but are not limited to apalutamide, bicalutamide, darolutamide, enzalutamide, flutamide, abiraterone acetate, ARN-509, and nilutamide.

Additionally or alternatively, in some embodiments, the kits further comprise at least one EGFRi that are useful for treating treating or preventing neuroendocrine tumor formation. Examples of EGFRis include, but are not limited to, osimertinib, afatinib, erlotinib, gefitinib, icotinib, dacomitinib, rociletinib, olmutinib, cetuximab, panitumumab, nimotuzumab, and necitumumab.

Additionally or alternatively, in some embodiments, the kits further comprise at least one chemotherapeutic agent that are useful for treating or preventing neuroendocrine tumor formation. Examples of chemotherapeutic drugs include, but are not limited to cyclophosphamide, fluorouracil (or 5-fluorouracil or 5-FU), methotrexate, edatrexate (10-ethyl-10-deaza-aminopterin), thiotepa, carboplatin, cisplatin, taxanes, paclitaxel, protein-bound paclitaxel, docetaxel, vinorelbine, tamoxifen, raloxifene, toremifene, fulvestrant, gemcitabine, irinotecan, ixabepilone, temozolmide, topotecan, vincristine, vinblastine, eribulin, mutamycin, capecitabine, anastrozole, exemestane, letrozole, risedronate, pamidronate, ibandronate, alendronate, denosumab, zoledronate, tykerb, anthracyclines (e.g., daunorubicin and doxorubicin), oxaliplatin, melphalan, etoposide, mechlorethamine, bleomycin, microtubule poisons, annonaceous acetogenins, abraxane, leucovorin, nab-paclitaxel, everolimus, pegylated-hyaluronidase, pemetrexed, folinic acid, MK2206, GDC-0449, IPI-926, M402, LY293111 or combinations thereof.

The kits may further comprise pharmaceutically acceptable excipients, diluents, or carriers that are compatible with one or more kit components described herein. Optionally, the above described components of the kits of the present technology are packed in suitable containers and labeled for the treatment or prevention of neuroendocrine tumors. The kits may optionally include instructions customarily included in commercial packages of therapeutic products, that contain information about, for example, the indications, usage, dosage, manufacture, administration, contraindications and/or warnings concerning the use of such therapeutic products.

### EXAMPLES

The present technology is further illustrated by the following Examples, which should not be construed as limiting in any way. For each of the examples below, any MEK inhibitor or CDK4/6 inhibitor described herein could be used.

### Example 1: General Methods and Procedures

### Study Design

The purpose of this study was to evaluate the potential efficacy of therapeutic targeting of exportin 1 (XPO1) at preventing neuroendocrine transformation and to investigate the underlying mechanism of action. Exportin 1 expression was analyzed in clinical specimens were analyzed at the mRNA and protein abundance, and isogenic cell lines were generated to study how early molecular alterations in the transformation process affect exportin 1 expression. Different preclinical xenograft models of neuroendocrine transformation were treated in vivo with exportin 1 inhibitors in combination with targeted therapy or chemotherapy to assess efficacy at controlling tumor growth. Endpoints for each treatment group were defined as the time when group average size would reach 1000 mm³ of volume. Where specified, tumors were harvested, and the expression of neuroendocrine and other markers of interest was assessed. All experiments were randomized and blinded where possible. Sample sizes were determined on the basis of expected effect sizes from pilot experiments. In general, group sizes of five or more mice were used. Differences in tumor growth were tested using Student's t-test (two-tailed), correcting for multiple measurements. All in vitro experiments were run at least in biological triplicates each including technical triplicates.

### Cell Lines

H1563 (CRL-5875) and H660 (CRL-5813) were purchased from ATCC. LnCap/AR and 22PC cell lines were kindly shared by Sawyers lab at MSKCC and maintained as previously described (RPMI+10% FBS++1% L-gulamine (Corning, #10-041-CM)+1% Sodium Pyruvate (Sigma, #S 8636-100ML), with the addition of 0.1 nM Dihydrotestosterone (Sigma, #521-18-6) for 22PC)⁷. Cell lines were authenticated through the STR characterization method and regularly tested for Mycoplasma (Universal Mycoplasma Detection Kit, #30-1012K, ATCC). All experiments were performed in low passage cells. All cell lines were cultured according to ATCC guidelines or as previously described^{7.}

TP53/RB1-deficient PRAD cell lines were generated as previously described⁷ with CRISPR/Cas9 technology. The CRISPR/Cas9 lentiviral vectors used to inactivate TP53 and RB1 genes were pLKO5.sgRNA.EFS.tRFP (Addgene, #57823) and lentiCRISPR v2 (Addgene, #52961), which were transduced into Cas9-expressing cell lines. TP53/RB1-deficient LUAD cell lines were generated by lentiviral transduction of a construct expressing a dominant negative TP53 isoform and a short hairpin RNA against RB1, produced from the FU-CYW vector that was previously described¹⁸ and kindly shared by Dr. Owen Witte. Other lentiviral overexpression plasmids used in this work included SOX2 (EX-T2547-Lv105-B), POU3F2 (EX-A3238-Lv151) and ONECUT2 (EX-Z4476-Lv151), purchased from Genecopoeia.

### Cell Cycle Assays

Cell cycle was studied by flow cytometry. Cells were seeded in 6-well plates and treated for 4 days with selinexor at the indicated doses. At day 4, cells were washed with PBS (Lonza) and fixed in 70% ethanol for a maximum period of 1 week at 4° C. Next, cells were washed twice with PBS (Lonza) and incubated with FxCycle PI/RNAse Staining solution (Invitrogen) for 1 h at RT. Cell cycle was determined using a BD LSRFortessa cell analyzer (BD Bioscience) and cell cycle phases were determined using FlowJo® software v10.

### Monotherapy Cytotoxicity Assay and in Vitro Treatments

Cytotoxic assays were performed as described in²² with a total of 1,500 cells/well seeded in 96-well plates and treated with the drugs/doses described for 96 hours. Viability was assessed with the CellTiter-Glo 2.0 Assay (Promega, G9242) as indicated by manufacturer, and normalized to the untreated control wells.

### Synergy Assays

Cells were seeded in 96-well plates (1500 cells/well) and treated with the interval of concentrations of cisplatin or selinexor for 5 days. Then, cell viability was assessed with CellTiter-Glo 2.0 Assay (Promega, G9242) and normalized to the untreated wells. Synergy was calculated using the ZIP method using the SynergyFinder web application (2.0)²³.

### Promoter Report Assays

A Promoter reporter clone for the human XPO1 gene (HPRM44900-LvPG04, Genecopoeia) was used in combination with a GAPDH positive control clone (GAPDH-LvPG04, Genecopoeia) and a negative control clone (NEG-LvPG04, Genecopoeia). Such clones were purchased in a lentiviral vector (LvPG04). Lentiviral particles were produced and used to infect isogenic cell lines of interest, as described previously(11), through concurrent transfection of HEK293T cells (ATCC, #CRL-1573) with a 3:2:1 ratio of lentiviral plasmid:psPAX2:pMD2.G with JetPrime transfection reagent (Polyplus, #114-15) at a 2:1 JetPrime: DNA ratio. Medium was changed 24 h after transfection and viral supernatants were collected 72 h after transfection. Viral supernatants were syringe-filtered with a 0.45-μM PVDF filter (Millipore, #SLHVM33RS) and concentrated approximately 20-fold with Lenti-X Concentrator (Takara Bio, #631232) according to the manufacturer's protocol. Promoter reporter assays were performed as specified by manufacturer using the Secrete-Pair Gaussia Luciferase Dual and Single Luminescence Assay Kits (LF032, Genecopeia), where signal from constitutively secreted alkaline phosphatase activity was used to normalized XPO1 promoter-dependent Gaussia Luciferase activity.

### Immunoblot

Protein extraction and western blot were performed as previously described²⁴ from frozen cell pellets or flash-frozen tumor samples using RIPA lysis buffer with 1×HALT protease inhibitor cocktail (Thermo, #78446). Cell pellets were resuspended in five volumes of cold lysis buffer and incubated on ice for 30 min. Lysates were clarified by centrifugation at 20,000 g for 10 min at 4° C. Antibodies for XPO1(#46249 , Cell Signaling Technology), pAKT (#4060, Cell Signaling Technology), pPRAS40 (#13175, Cell Signaling Technology), Chromogranin A (#ab85554, abcam), synaptophysin (#36406, Cell Signaling Technology), CD56 (#99746, Cell Signaling Technology), AR (#5153, Cell Signaling Technology), POU3F2 (#12137, Cell Signaling Technology), ONECUT2 (ab28466, Abcam), vinculin (#13901, Cell Signaling Technology) and tubulin (#3873, Cell Signaling Technology). Quantifications were performed with the Image Studio software (Version 3.1, Li-Cor). Antibodies used for IHC included exportin 1 (#611833 from BD), synaptophysin (Dako #A0010), and chromogranin A (Dako #A0430). IHC was performed on FFPE tissue from resected tumor samples obtained from patients with de novo and transforming LUAD, SCLC, PRAD and NEPC. For immunohistochemical staining, slides were deparaffinized and steamed for 45 min in Target Retrieval Solution (Dako). Immunocomplexes were detected using PV Poly-HRP anti-mouse IgG (Leica Microsystems, #PV6114) followed by a TSA biotin amplification step (Perkin Elmer) with DAB as the chromogen. Tissue sections were counterstained with hematoxylin, and slides were digitized on a Ventana DP 200 Slide Scanner (Roche). Expression was scored in a blinded manner by pathologists, whereby the optical density level (“0” for no brown color, “1” for faint and fine brown chromogen deposition “2” for intermediate chromogen deposition and “3” for prominent chromogen deposition) was multiplied by the percentage of cells at each staining level, resulting in a total H-score range of 0-300. All study subjects had provided signed informed consent for biospecimen analyses under Institutional Review Board-approved protocol.

### In Vivo Treatments

4-10 female (22PC, PDXs) or male (LnCap/AR) 6-week old NOD. Cg-Prkdc<scid>Il2rg<tm1Wjl>/SzJ (NSG) mice (PDXs) or female 6-week-old athymic nude mice (cell line xenografts) were subcutaneously engrafted per treatment arm and until tumors reached 100-150 mm³. At that point, mice were randomized into groups and treated with either vehicle, cisplatin (2 mg/kg intraperitoneally (i.p.) once/week), etoposide (3 mg/kg i.p. QDx3), selinexor (10 mg/kg p.o. QDx3), enzalutamide (10 mg/kg per os (p.o.) QDx5), osimertinib (25 mg/kg p.o. QDx5) or the combinations of cisplatin+etoposide, cisplatin+selinexor, enzalutamide+selinexor or osimertinib+selinexor at the previously mentioned doses. Mice weights and tumor volumes were measured twice a week and mice were sacrificed when tumors reached humane endpoint (volume=1000 mm³). The number of mice per treatment arm were selected according to previous experience with the models and response to treatments. Blinding was not performed. All animal experiments were approved by the Memorial Sloan Kettering Cancer Center (MSKCC) Animal Care and Use Committee.

### RNA Extraction

Frozen tissues or cell pellets were weighed and homogenized in RLT and nucleic acids were extracted using the AllPrep DNA/RNA Mini Kit (QIAGEN, #80204) according to the manufacturer's instructions. RNA was eluted in nuclease-free water.

### RNAseq Alignment and Quantification

Transcript abundances were quantified using RNA-seq reads by Salmon v1.1.0²⁵ Raw reads of RNA-seq were mapped to 25 mer indexed hg 38 genome. In addition to default settings, mapping validation (--validatemappings), bootstrapping with 30 re-samplings (numBootstraps), sequence specific biases correction (--seqBias), coverage biases correction (--posBias) and GC biases correction (--gcBias) were also enabled. Transcripts were mapped to genes based on Ensembl 92²⁶, normalized by size factor at gene level. Subsequently the differential gene expression were evaluated on Salmon output files using Sleuth v0.30.0²⁷ in gene mode. Wald test was performed on differential gene expressions. Genes were marked as significantly differentially expressed if the False Discovery Rates, q, calculated using the Benjamini-Hochberg method, was less than 0.05, and beta (Sleuth-based estimation of log2 fold change)>0.58, which approximately equivalent to a log2 fold change of 1.5.

### Publicly Available RNAseq Datasets Analyses

Public datasets leveraged in the present manuscript accessible through cbioportal. com include Abida et al., PNAS 2019, LUAD TCGA PanCancer, LUAD OncoSG, Nat Gen 2020 and PRAD TCGA PanCancer. The dataset from Tsai et al., BMC Cancer 2017 can be accessed at Gene Expression Omnibus portal (GSE104786). The public sets were divided into four groups according to their mutation status of TP53 and RB1, as the following, TP53WT/RB1WT, TP53MT/RB1WT, TP53WT/RB1MT, and TP53MT/RB1MT. RNAseq expression distribution of XPO1, and SOX2 were presented in box plots for the above four groups of samples. RNAseq expression values were downloaded through cBioPortal. <data type 1:RSEM> The expression levels for LUAD (OncoSG, Nat Genet 2020) are in RSEM (RNAseq by Expectation-Maximization) that have been normalized using DESeq2 v.1.16.1 followed by log transformation whereas that for PRAD (TCGA, PanCancer) are in batch normalized RSEM then followed by log transformation. <data type 2: RSEM z-score> Log-transformed mRNA expression z-scores compared to the expression distribution of all samples were downloaded for both LUAD (OncoSG, Nat Genet 2020)²⁸ and PRAD (TCGA, PanCancer)²⁹. The pairwise comparisons of mean expressions were conducted among previously mentioned four groups and evaluated by Wilcoxon test. (Using traditional RNAseq DEG approach to evaluate DE p value by limma pipeline: We applied linear modelling on the normalized and log transformed RSEM values which are assumed to be normally distributed using limma (v3.28.14)³⁰. The coefficients and standard errors were then estimated for each pair of contrast from the linear model. Empirical Bayes Statistics for differential expressions were carried out to evaluate the significance value.)

The expression values of XPO1 and SOX2 were correlated in scatter plots for previously mention seven cohorts. RNAseq expression values were downloaded through cBioPortal. <data type 1:RSEM> The expression levels are in RSEM (RNAseq by Expectation-Maximization) that have been using DESeq2 v.1.16.1 normalization, LUSD (OncoSG, Nat Genet 2020)²⁸, or batch normalized followed by log transformation. <data type 2: RSEM z-score> Log-transformed mRNA expression z-scores compared to the expression distribution of all samples were downloaded. The expression correlations were evaluated by Pearson (Spearman).

### Pathway Enrichment Analyses

Gene set enrichment analysis (GSEA)³¹ was conducted on the full sets of differential gene expression output from the previously mentioned comparisons. Genes were ranked by p value scores computed as −log10(p value)*(sign of beta). The annotations of gene set were taken from Molecular Signatures Database (MSigDB v7.0.1)^{31,32} of gene set enrichment was evaluated using permutation test and the p value was adjusted by Benjamini-Hochberg procedure. Any enriched gene sets with adjusted p value≤0.1 were regarded as significant. This analysis was conducted using ClusterProfiler R package v3.18.1³³. Some enriched gene sets of interests were selected and their pathway annotations were concatenated manually to remove redundancy and achieve high level generality. When the pathway terms were merged, median enrichment score was taken as the new group enrichment score, p values were aggregated using Fisher's method from the Aggregation R package³⁴, and core enrichment of genes were collapsed. The consolidated gene sets enrichment were then presented in dot plots.

### ATAC-Seq

The reads were trimmed for both quality and Illumina adaptor sequences using trim_galore v0.4.4 (github.com/FelixKrueger/TrimGalore) in the pair-end mode. Then the raw reads were aligned to human assembly hg38 using bowtie2 v2.3.4³⁵ using the default parameters. Aligned reads with the same start site and orientation were removed using the Picard tool (broadinstitute.github.io/picard/). Enriched regions in individual samples were called using MACS2³⁶ and then filtered against genomic ‘blacklisted’ regions (http://mitra.stanford.edu/kundaje/akundaje/release/blacklists/hg38-human/hg38.blacklist.bed.gz). The filtered peaks within 500 bp were merged to create an union of peak atlas. Raw read counts were tabulated over this peak atlas using featureCounts v1.6.0³⁷. The read counts were then normalized with DESeq2. The read density profile in the format of bigwig file for each sample was created using the BEDTools suite (bedtools.readthedocs.io) with the normalization factor from DESeq2³⁸. All bigwig genome tracks on XPO1 gene region were generated using pyGenomeTracks v3.5³⁹.

### RT-qPCR

Retrotranscription was performed with the Superscript IV VILO kit (Fisher Scientific, #11756050) following manufacturer's instructions. The following TaqMan (ThermoFisher) probes were used: CHGA (Hs00900370_m1), INSMI (Hs00357871_s1) and ASCLI (Hs00269932_m1), XPO1 (Hs00185645_m1), SOX2 (Hs04234836_s1) and UBC (Hs00824723_m1). Data was analyzed as previously described²².

### Clinical Samples

All study subjects had provided signed informed consent for biospecimen analyses under an Institutional Review Board-approved protocol. Metastatic prostate cancer samples were collected as part of the Prostate Cancer Donor Program at the University of Washington. Tissue microarrays sampling PRAD and NEPC formalin fixed paraffin embedded tissues were used in this study.

### Statistical Analyses

Comparisons between two groups were performed using paired or unpaired two-tailed Student's t test, as indicated in Fig. legends. A p value <0.05 was considered statistically significant (*p≤0.05, **P≤0.01 and ***P≤0.001). N indicates the number of biological replicates, all bars within the graphs represent mean values, and the error bars represent SEMs or standard deviation, as indicated in the Fig. legend. All in vitro experiments were replicated a minimum of 3 times (biological replicates). All western blots have been replicated a minimum of 2 times (biological replicates). Please refer to previous sections for detailed statistical analyses of the bioinformatic data.

### Example 2: Exportin 1 is Upregulated During NE Transformation

To evaluate the potential role of XPO1 in NE transformation, we first assessed expression across a previously published transcriptomic dataset on NE-transforming clinical specimens⁶ including LUADs that never transformed (categorized as “LUAD”); LUADs obtained from pre-transformation or microdissected combined histology cases, thought to be derived from lineage plasticity (categorized as “T-LUAD”); SCLCs from post-transformation or microdissected combined histology cases (“T-SCLC”), and de novo SCLCs (“SCLC”).

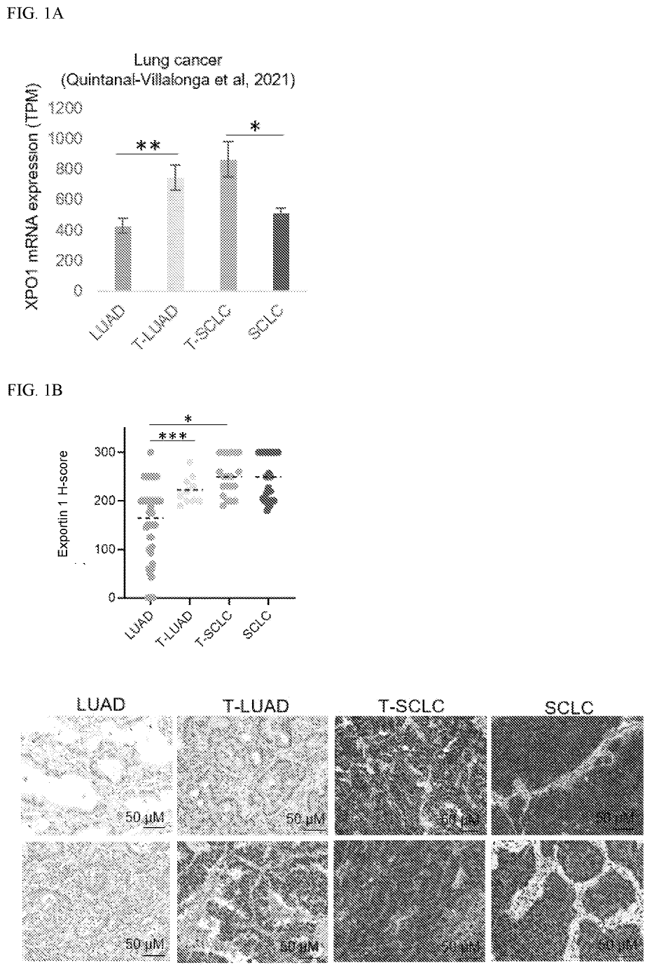

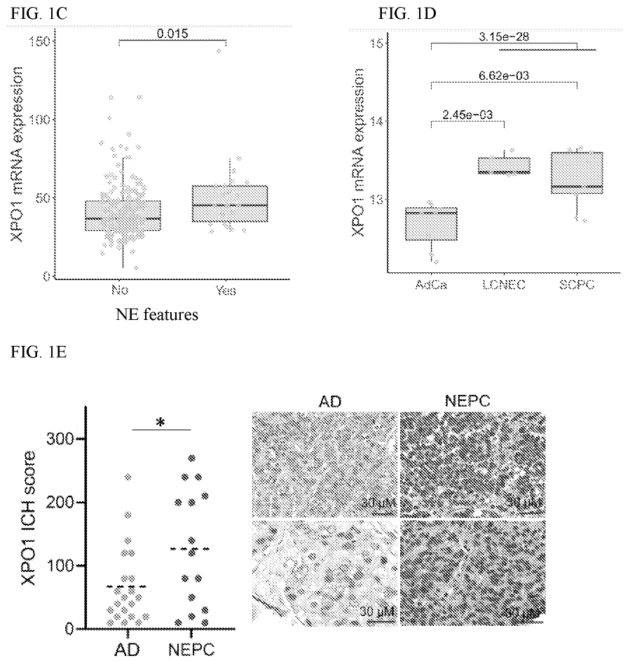

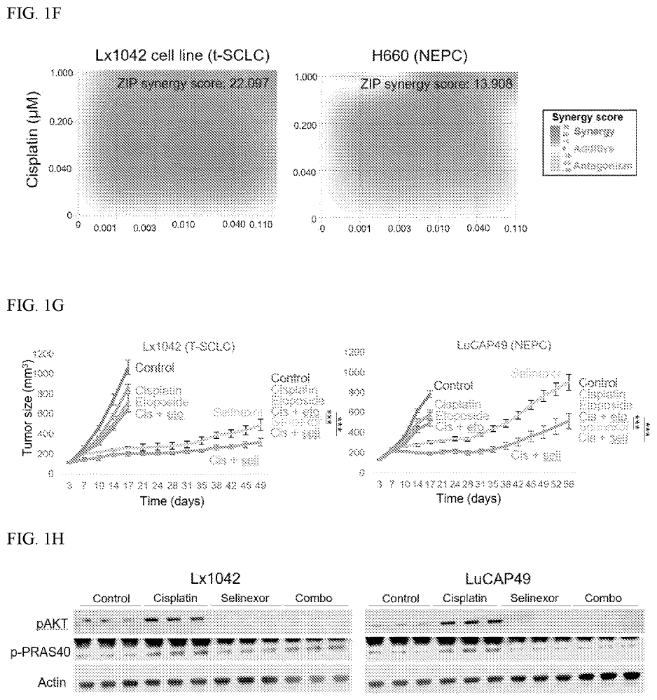

Exportin 1 is more highly expressed in SCLC clinical specimens than in any other tumor type¹¹, suggestive of a dependency for small cell carcinomas to this nuclear exporter. Assessment of XPO1 mRNA expression levels in a previously described cohort of LUAD-to-SCLC transforming clinical specimens⁶ revealed upregulation of XPO1 upon NE transformation (FIG. 1A). Consistently, determination of exportin 1 protein levels by immunohistochemistry in an independent cohort of transforming lung cancer clinical specimens and patient-derived xenografts (PDXs) confirmed increased exportin 1 protein expression in transforming LUADs (T-LUADs) versus control, never-transforming LUADs, and further upregulation upon transformation to transformed SCLC (T-SCLC, FIG. 1B). Although XPO1 mRNA expression was higher in T-SCLC as compared to de novo SCLC (FIG. 1A), protein abundance of exportin 1 was comparable in these cohorts (FIG. 1B).

Additionally, by leveraging publicly available prostate carcinoma clinical specimen datasets^{14,15}, we observed that the presence of NE features is associated to increased XPO1 mRNA levels in prostate AD (PRAD, FIG. 1C), and that prostate tumors with NE differentiation, including large cell NE and small cell prostate carcinomas, show increased XPO1 expression as compared to PRAD (FIG. 1D). In line with these results, assessment of exportin 1 protein levels in prostate carcinomas revealed higher expression of the gene in NEPC as compared to PRAD (FIG. 1E). These results suggest that NE carcinomas may be particularly dependent on exportin 1 function, and high expression of XPO1/exportin 1 at stages temporally proximal to NE transformation are consistent with a role in promoting histologic transformation.

### Example 3: The Combination of Exportin 1 Inhibition and Chemotherapy is Effective Against NE-Transformed Carcinomas

Studies have shown that inhibition of exportin 1 dramatically sensitizes PDXs derived from de novo SCLCs to chemotherapeutic agents used in the first and second line treatment of these tumors, and that these effects are mediated by the suppression of AKT/mTOR signaling, which is induced in de novo SCLCs after treatment with chemotherapeutic agents, by exportin 1 inhibition¹¹. With increasing reports highlighting the molecular and treatment response differences between de novo and transformed SCLCs^{6,16} and suggesting that transformed SCLCs may retain molecular features of their previous LUAD state^{6,10}, such as decreased neuronal differentiation or increased notch signaling, the capacity of selinexor to sensitize transformed SCLCs and NEPCs to cisplatin was examined, a chemotherapeutic agent used in combination with etoposide in NE-transformed tumors. In vitro synergy assays in a cell line derived from a T-SCLC PDX (Lx1042) and in H660, a NEPC cell line demonstrated synergistic growth inhibition between selinexor and cisplatin (FIG. 1F). Consistently, in vivo treatments of PDXs derived from a T-SCLC (Lx1042) and a NEPC (LuCAP49¹⁷) engrafted subcutaneously in immunosuppressed NOD. Cg-Prkdcscid Il2rgtm1Wjl/SzJ (NSG) mice confirmed that the combination of selinexor and cisplatin outperformed the combination of cisplatin and etoposide (FIG. 1G), with treatment/control relative volume (T/C) values of 14% and 19% (cisplatin+selinexor versus control) and of 27% and 32% (cisplatin+selinexor versus cisplatin+etoposide) for Lx1042 and LuCAP49, respectively. Similarly to what was observed for de novo SCLCs¹¹, these NE-transformed models showed increased activation of the AKT pathway after cisplatin treatment, which was suppressed by selinexor (FIG. 1H), supporting an overlapping mechanism of cisplatin sensitization by selinexor in de novo and NE-transformed tumors. These results extend the potential use of selinexor in combination with chemotherapy to transformed SCLCs, as well as to NE-transformed prostate tumors.

### Example 4: TP53 and RB1 Loss Induce XpO1 Expression and Sensitivity to Exportin 1 Inhibition

Exportin 1 upregulation may occur early in the NE transformation process, with T-LUADs showing increased exportin 1 mRNA and protein expression than control, never-transforming LUADs (FIGS. 1A-1B). One of the hallmarks of NE transformation is the occurrence of TP53 and RB1 function loss, occurring through genomic alterations or protein downregulation^{4,6,10}. Importantly, inactivation of these two tumors suppressors occurs early in the transformation process and seems to be necessary, even if not sufficient, for histological transdifferentiation to occur^{4,10}. Thus, the loss of function of TP53 and RB1 on exportin 1 expression was assessed.

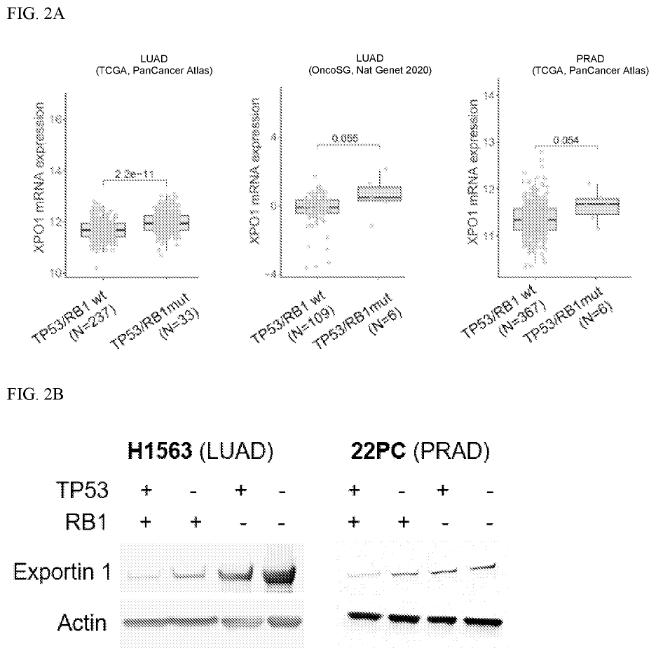

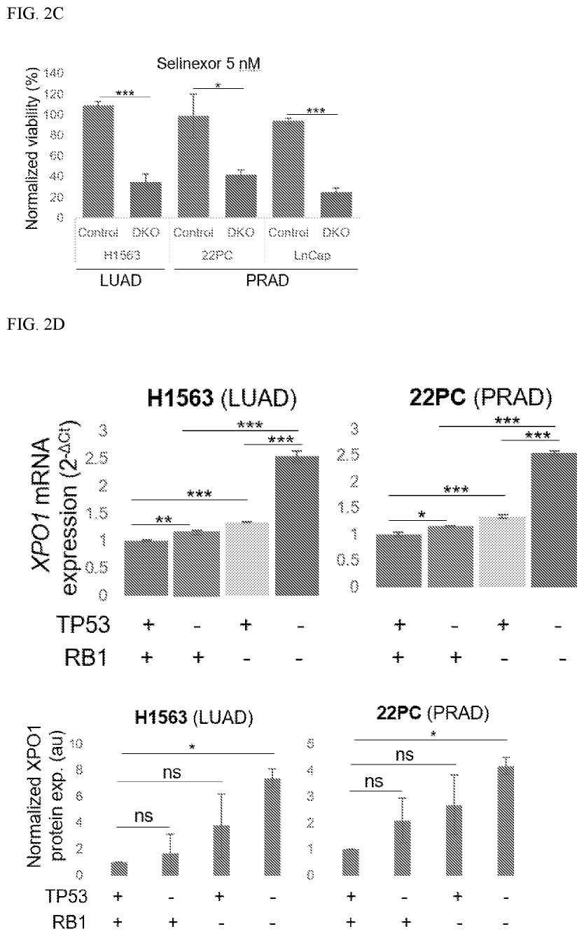

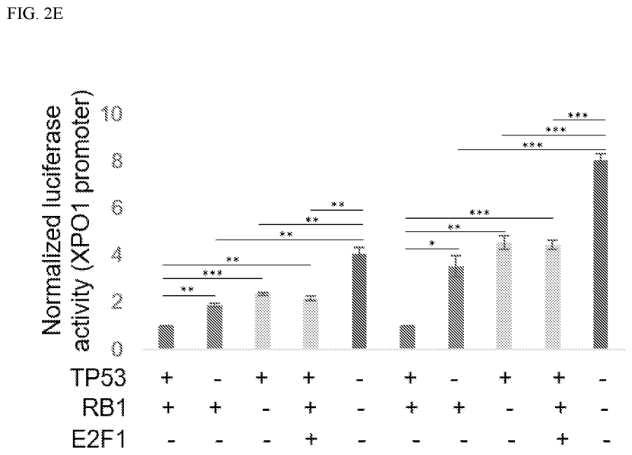

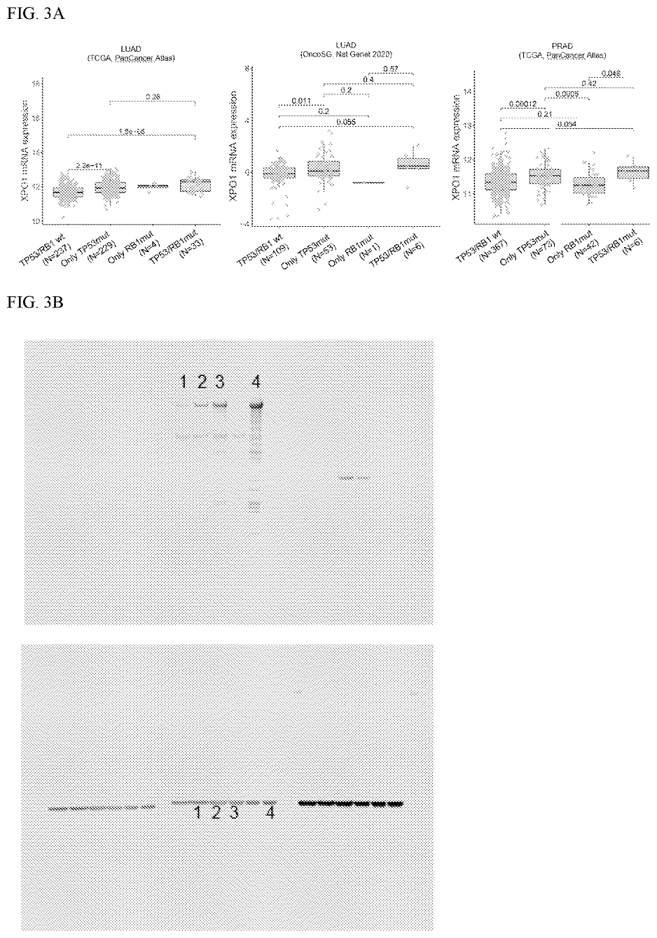

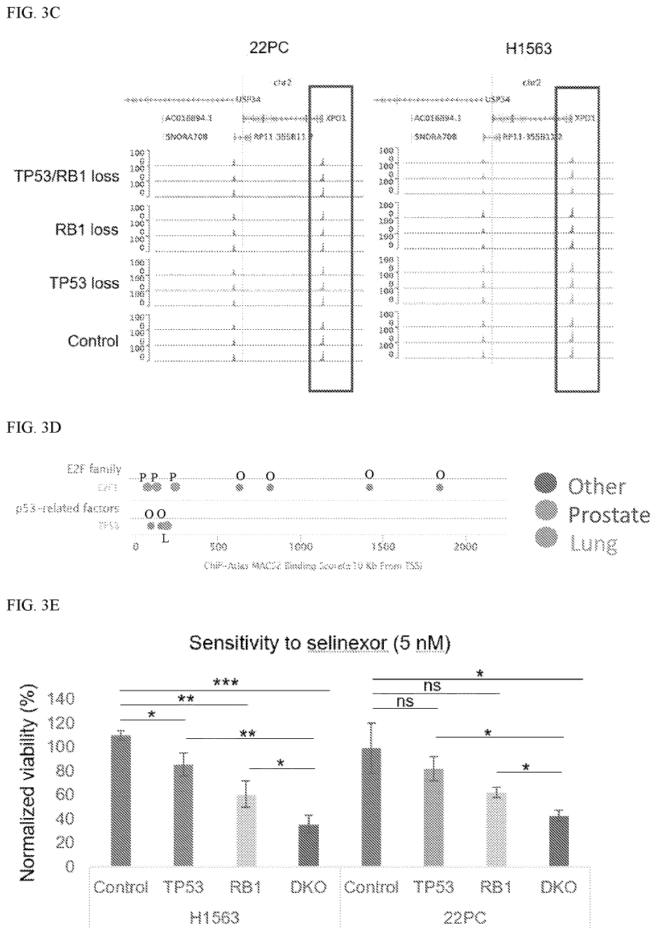

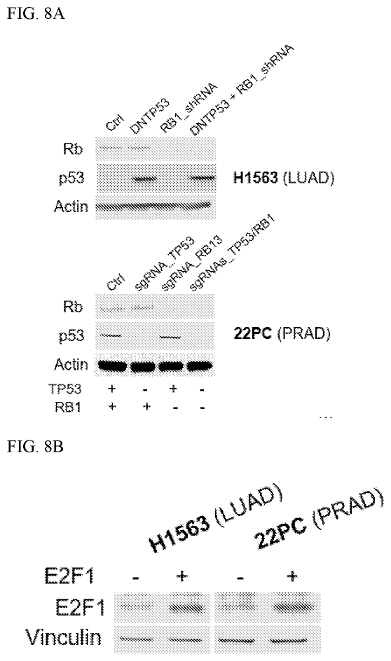

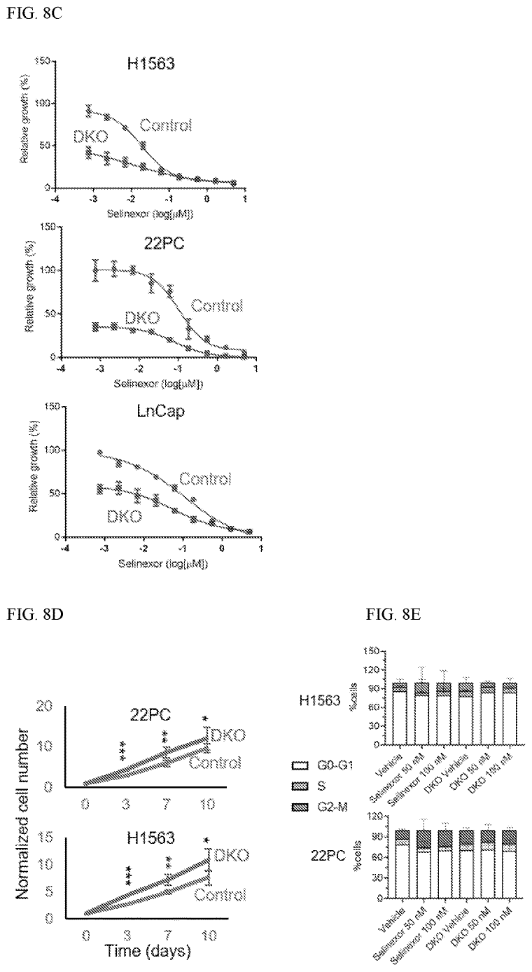

Consistent with this hypothesis, increased expression of XPO1 mRNA in LUADs and PRADs across multiple cohorts with concurrent TP53 and RB1 mutations was observed, as compared to their double wild-type counterparts (FIG. 2A). The assessment of the potential contribution of mutations on each of the genes individually was challenging due to the low number of samples showing RB1 genomic alterations without co-occurring TP53 mutations (FIG. 3A), but the data available showed an association of TP53 mutations to increased XPO1 expression as compared to TP53/RB1-wild type samples, even if double TP53/RB1-mutated samples showed the highest XPO1 mRNA levels (FIG. 3A). Then, TP53- and/or RB1-genetically inactivated LUAD (H1563, overexpression of dominant negative TP53 and short hairpin RNA against RB1) and PRAD (22PC, CRISPR-Cas9 inactivation) isogenic cell lines were generated and exportin 1 mRNA and protein expression were assessed by qPCR and western blot, respectively (FIGS. 2B, 2D and FIG. 8A). Loss of function of either gene induced exportin 1 expression on both cell lines compared to the control condition, and further upregulation was observed in the TP53/RB1-inactivated cell lines (FIGS. 2B, 2D), consistently to the clinical specimen data (FIG. 2A). Assay for Transposase-Accessible Chromatin (ATAC)-seq data on these TP53/RB1-inactivated cell lines did not reveal increased XPO1 gene accessibility after TP53/RB1 loss of function (FIG. 3C). However, leveraging publicly available ChIP-seq datasets, we observed that both TP53 and E2F Transcription Factor 1(E2F1), one of the major transcription factor effectors activated upon RB1 inactivation, bind to the transcription start site of the XPO1 gene in a number of datasets including prostate and lung specimens (FIG. 3D). Without wishing to be bound by theory, it is believed that TP53 and E2F1 might be directly regulating XPO1 transcription by binding to the XPO1 gene promoter. To test this, we performed promoter reporter assays for the XPO1 gene promoter, in isogenic cell lines derived from H1563 and 22PC (FIG. 2E).

In both cell lines, inactivation of TP53 led to increased XPO1 promoter activity, suggesting that TP53 binding could repress XPO1 gene expression. Similarly, inactivation of RB1 led to increased XPO1 promoter activity, at comparable amounts to E2F1 overexpression (FIGS. 3D and 8B), suggesting that E2F1 might be able to induce XPO1 gene expression following RB1 inactivation. Concurrent inactivation of TP53 and RB1 further increased XPO1 promoter activity (FIGS. 2A, 2D). Altogether, these data suggest that TP53 might directly repress XPO1 gene expression by directly binding the XPO1 promoter, whereas E2F1 might be able to directly bind the XPO1 gene promoter to induce XPO1 transcription after RB1 inactivation.

Additionally, treatment of matched isogenic cell lines, including an additional PRAD line (LnCap/AR), confirmed increased selinexor sensitivity in those cell lines with TP53/RB1 inactivation relative to either wild-type controls (FIGS. 2C and 8C) or single gene loss-of-function counterparts (FIG. 3E). To study if these effects may just be derived of TP53 and RB1 inactivation causing a more highly proliferative phenotype in these models, we performed proliferation assays in TP53/RB1-inactivated cell lines and studied the cell cycle profile in those being treated with selinexor (FIGS. 8D-8E). We observed a slight increase in proliferation after inactivation of TP53 and RB1, with no significant shifts in cell cycle profile even at substantially cytotoxic concentrations of selinexor. Thus, the induced selinexor sensitivity by TP53 and RB1 inactivation did not appear to be a consequence of substantial cell cycle disruption as might occur with non-specific cytotoxics. Altogether, these results suggest that loss of TP53 and RB1 activity may induce a higher dependency of the LUADs and PRADs on exportin 1.

### Example 5: Exportin 1 Inhibition Interferes With Ne Relapse on Targeted Therapy

The observed induction of exportin 1 and selinexor sensitivity upon concurrent TP53/RB1 loss (FIGS. 2A-2E and 3D) provided a rationale to nominate exportin 1 as a potential therapeutic target to prevent NE transformation in both the lung and prostate settings.

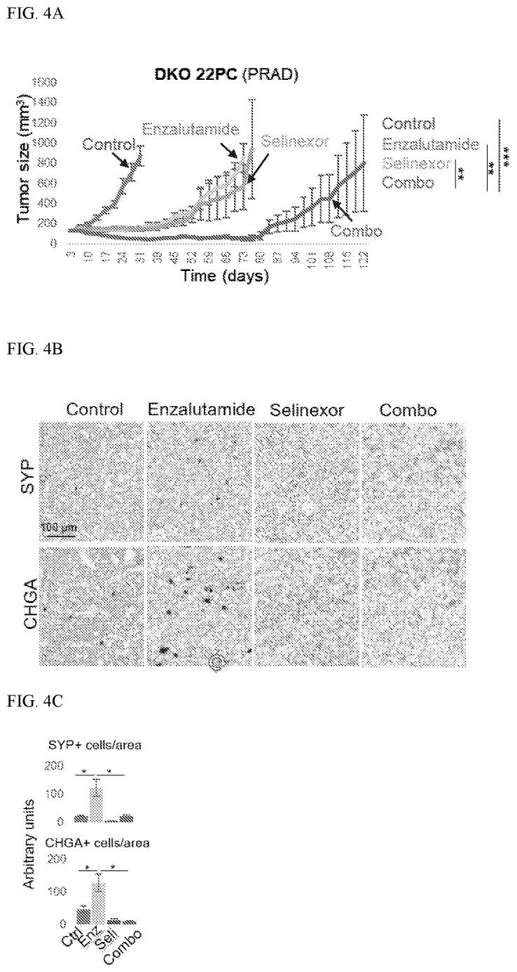

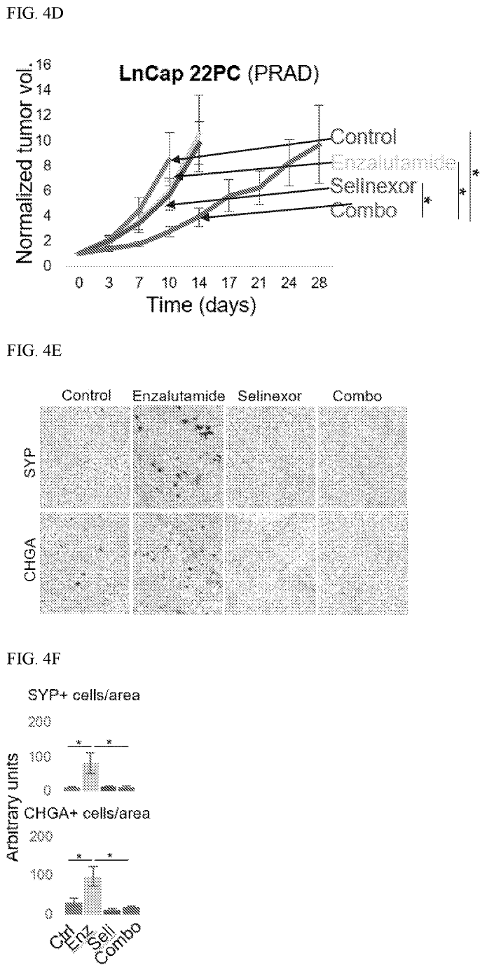

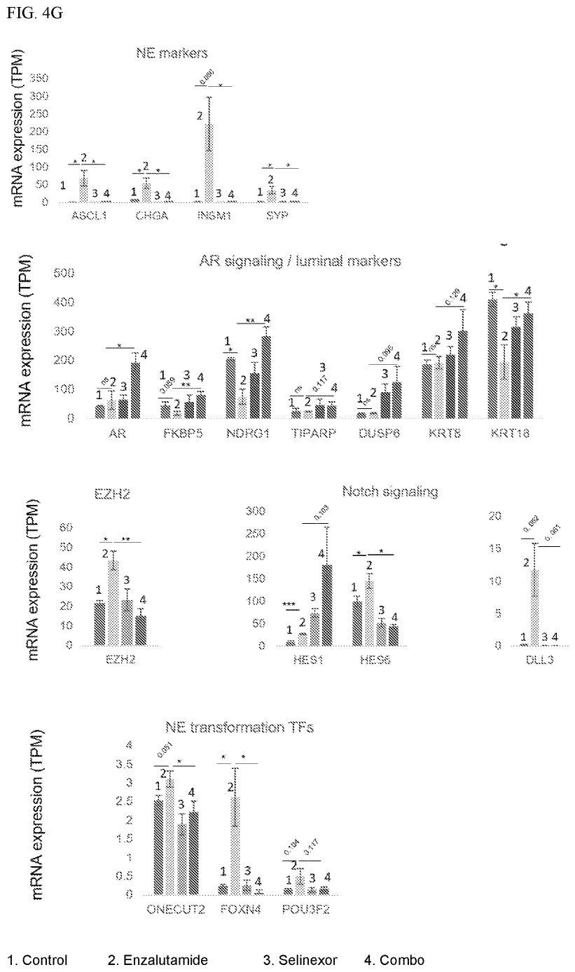

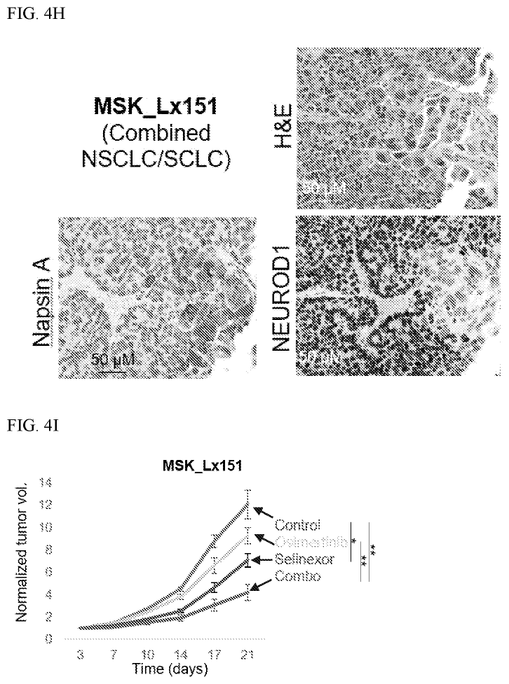

To test this hypothesis, we leveraged a previously described prostate NE transformation model⁷, the PRAD cell line 22PC and LnCap/AR, in which double knock out (DKO) of TP53 and RB1 induces Androgen Receptor (AR)-targeted therapy resistance in vivo, together with a loss of epithelial features and increased NE marker expression⁷. We generated xenografts using both cell lines in immunocompromised NSG mice, which were then treated with enzalutamide, selinexor or their combination by oral gavage after tumors reached around 100 mm³ (FIGS. 4A-4B).

Although initially responsive to enzalutamide (T/C values of 18% at control arm endpoint, defining endpoint for a given treatment group as the time when group average size would reach 1000 mm³ of volume), the DKO 22PC tumors consistently demonstrated acquired resistance within the first 2 months of treatment (FIG. 4A). These tumors also showed high initial sensitivity to selinexor monotherapy (T/C values of 17% at control arm endpoint), leading to resistance with a similar timing to that observed for enzalutamide (FIG. 4A). Combination treatment showed greater durability of response, approximately doubling time to tumor relapse relative to either drug alone (FIG. 4A). In the DKO LnCap/AR model, xenografts exhibited immediate resistance to either enzalutamide or selinexor monotherapies (FIG. 4B), with T/C values of 74 and 63%, respectively at control arm endpoint. In this model, again the combination of enzalutamide and selinexor exhibited the greatest effectivity at suppressing tumor growth (T/C value of 31% at control arm endpoint), doubling the time until relapse as compared to enzalutamide monotherapy arm (FIG. 4B).

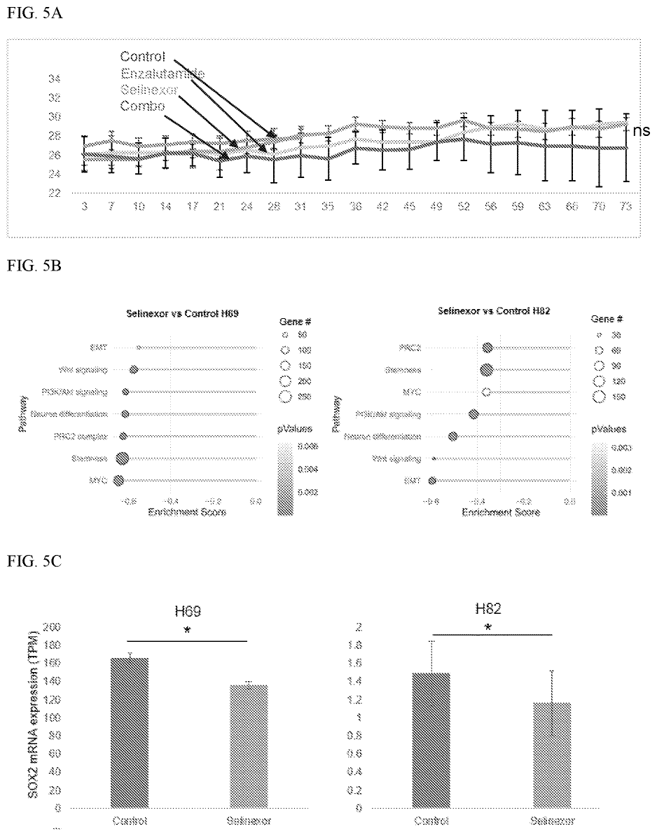

Remarkably, combination treatment did not show increased toxicity relative to enzalutamide monotherapy as determined by mouse body weight over time (FIG. 5A).

Determination of protein expression of NE markers synaptophysin and chromogranin A by IHC in the tumors collected at experimental endpoint for each of the different treatment arms revealed an increased NE cell subpopulation in the enzalutamide-treated versus the control tumors in both models (FIGS. 4B-4F). This was consistent with the previously described ability of targeted therapy to induce and enrich for histology transdifferentiated cells^{6,9}. Interestingly, we observed reduced representation of this cell subset in the selinexor- and combo-treated arms (FIGS. 4B-4F), suggesting that exportin 1 inhibition can inhibit NE transformation. To further dissect this phenotype, we performed transcriptome sequencing in tumors from all treatment arms collected at control arm endpoints for the DKO 22PC model (day 31, FIG. 4A). In line with the IHC data, we observed increased expression of the NE markers ASCL1 (p=0.046), CHGA (p=0.039) and SYP (p=0.040) in the enzalutamide-treated tumors (FIG. 4G), an effect that was reverted by selinexor in the combo-treated group.

In addition to classical NE marker expression, we observed a transcriptomic profile compatible with NE transformation in the enzalutamide-treated tumors, characterized by increased expression of (1) HES6 and DLL3, markers of Notch signaling downregulation; (2) EZH2, the catalytic component of the PRC2 complex, an epigenetic remodeling complex involved in lineage plasticity and histological transformation^{6,8}, and (3) transcription factors previously involved in NE transformation including ONECUT2, FOXN4 and POU3F2 (FIG. 4G). This NE-transformation transcriptomic signature was suppressed by selinexor in the combination-treated tumors, which also exhibited higher maintained expression of AR, AR targets such as FKBP5 or NDRG1, and of luminal markers such as KRT8 and KRT18 (FIG. 4G).

Additionally, we observed that selinexor prevented the acquisition of a basal-like phenotype, previously described to be potentiated in TP53/RB1-deficient PRAD after targeted therapy treatment, in parallel with NE features(9) (FIG. 5D), further supporting the capacity of exportin 1 inhibition to constrain lineage plasticity in this setting.

A parallel model of in vivo LUAD-to-SCLC transformation has not been described. However, we identified a PDX (MSK_Lx151) derived from an EGFR-mutant mixed histology tumor which retained both LUAD and SCLC components in the mouse (FIG. 4H), potentially representative of an intermediate state of transformation. In this PDX model both osimertinib and selinexor demonstrated limited efficacy as single agents, with T/C values of 73.30% and 57.67%, respectively, at control arm endpoint (FIG. 4I).

Again, the combination of Osimertinib and selinexor demonstrated greater efficacy, with a T/C value of 34.15% at control arm endpoint and no additional toxicity compared to osimertinib monotherapy (FIG. 5E). No significant differences were observed in the tumors collected at the endpoint of each of the treatment arms under study in terms of expression of the LUAD marker TTF-1 and the NE markers synaptophysin and chromogranin A (FIG. 5F), suggesting that the combination of osimertinib and selinexor may not be able to revert NE transformation after it has occurred.

Altogether, these results demonstrate that exportin 1 inhibition suppresses the acquisition of a NE phenotype in models of NE transformation, and that its combination with targeted therapy are useful in preventing, or delaying NE relapse in LUADs and PRADs prone to transformation.

### Example 6: Exportin 1 Inhibition Blocks SOX2 Induction in Models of NE Transformation

To study the mechanism by which exportin 1 inhibition was able to interfere with NE relapse in our models, we performed differential gene expression (DEG) and pathway enrichment analyses on the transcriptomic data available for the DKO 22PC transformation model (FIGS. 4A, 4C, 4E, 4G).

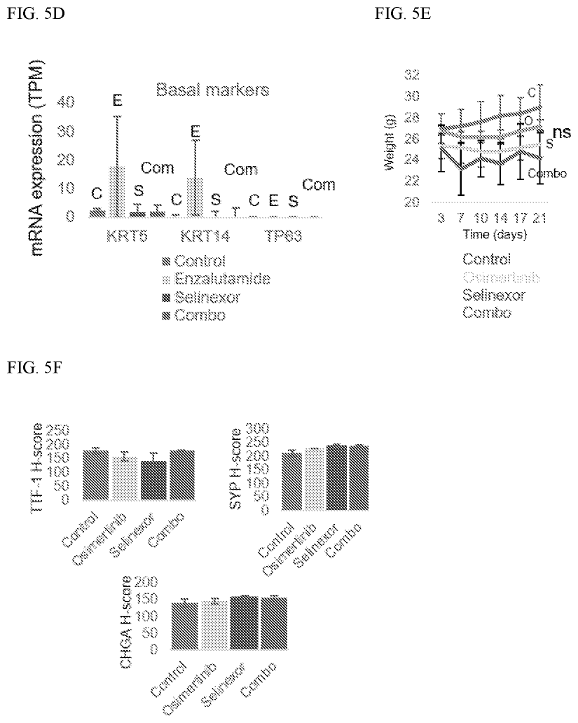

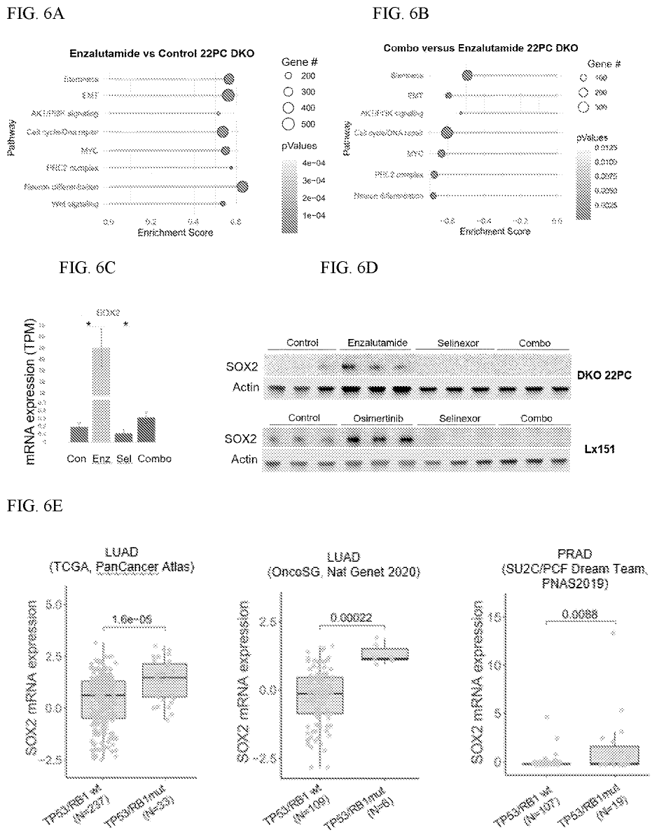

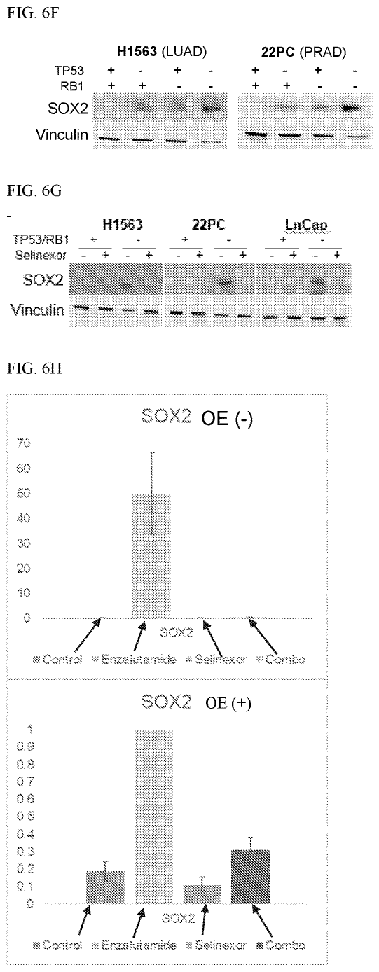

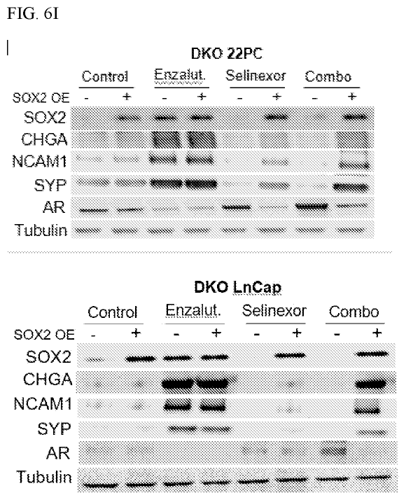

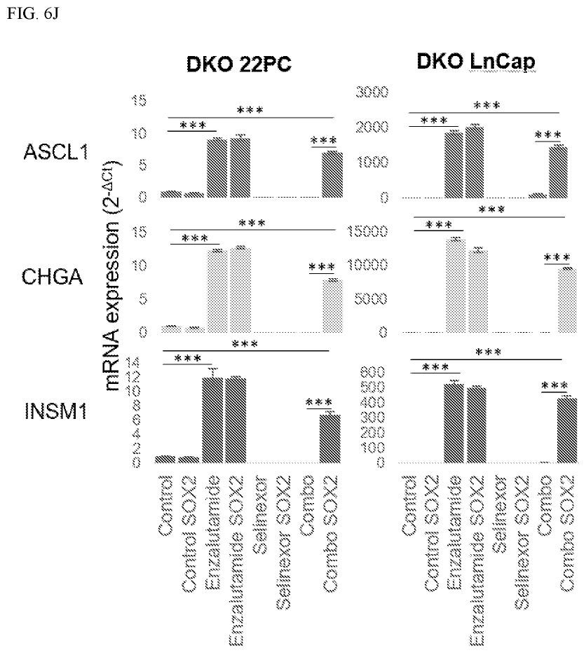

In the comparison of enzalutamide-treated versus control tumors, we observed upregulation of genes involved in several pathways previously implicated in NE transformation^{5,6,18}, including pathways related to epithelial-to-mesenchymal transition, stemness, PRC2 complex, and AKT and Wnt signaling, among others (FIG. 6A). When comparing the combo-versus enzalutamide-treated tumors, we observed downregulation of all these pathways when selinexor was added (FIG. 6B).

Importantly, leveraging previously published¹¹ transcriptomic data on selinexor-treated de novo SCLC cell lines, we observed downregulation of these pathways as well (FIG. 5B), further supporting the ability of exportin 1 inhibition to interfere with these NE transformation-related pathways.

SOX2, a transcription factor implicated in maintenance of stem cell capacity, was found to be essential for NE transformation in a prostate cancer model⁷, and is highly overexpressed in SCLC. SOX2 expression was induced by enzalutamide in the TP53/RB1-inactivated (DKO) 22PC model, and this induction was inhibited by selinexor in the combination-treated group (FIG. 6C). We observed SOX2 protein upregulation induced by targeted therapy in tumors from both DKO 22PC and Lx151 models, again suppressed by the addition of selinexor (FIG. 6D), and SOX2 mRNA downregulation in de novo SCLC cell lines treated with selinexor¹¹ (FIG. 5C).

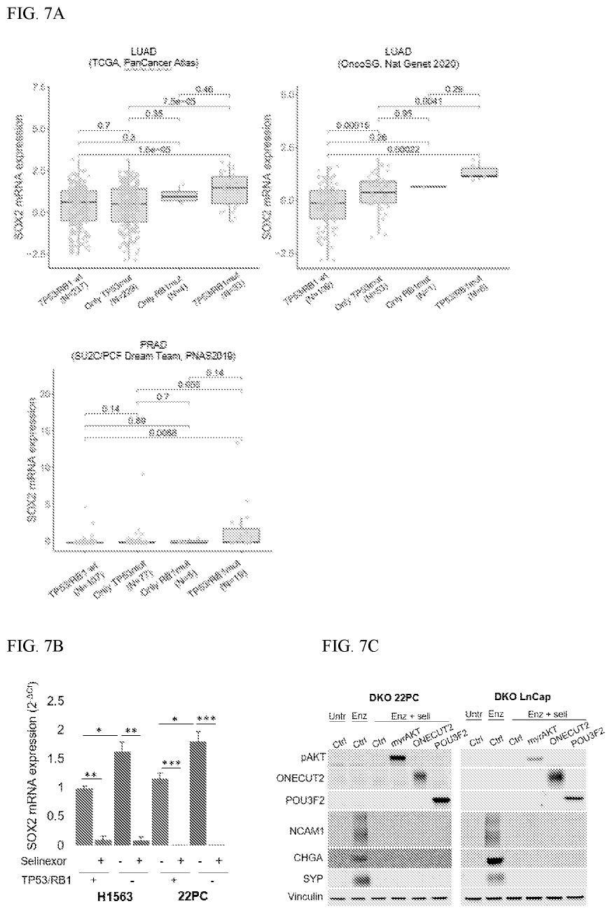

Upregulation of SOX2 expression has been described to start early in the transformation process in preclinical prostate models of NE transformation, and concretely after inactivation of TP53 and RB1⁷. Consistent with these preclinical observations, SOX2 mRNA expression was upregulated in double TP53/RB1-mutant LUAD and PRAD clinical specimens, as compared to their double wild-type counterparts (FIG. 6E and FIG. 7A).

SOX2 mRNA and protein expression was also elevated in isogenic TP53/RB1-inactivated LUAD and PRAD cell lines (FIGS. 6F and 7B).

This induction was prevented by selinexor treatment at both mRNA and protein levels (FIGS. 6G and 7B). Previous reports have implicated miR-145 in the downregulation of SOX2 mediated by XPO1 inhibition. We were not able to detect miR-145 expression in any of the lung or prostate adenocarcinoma samples under study. Altogether these data supported the hypothesis that exportin 1 inhibition might interfere with NE transformation in TP53/RB1-deficient adenocarcinomas by preventing SOX2 upregulation in early steps of transformation, consistent with a prior report that ectopic SOX2 suppression prevented induction of NE features in PRAD models and maintained enzalutamide sensitivity (9). To test this hypothesis, we exogenously overexpressed SOX2 in the DKO PC22 and LnCap/AR NE transformation models and treated as previously with enzalutamide, selinexor, or their combination, with the aim to characterize changes in NE marker and AR expression in vitro (FIGS. 6I-6J). In the untreated condition, SOX2 ectopic overexpression did not alter expression of either, suggesting that even if SOX2 is required for NE transformation, its sole overexpression in a TP53/RB1-deficient background may not be enough to induce a NE phenotype. As observed in vivo, enzalutamide-treated cells exhibited high SOX2 expression (FIG. 6H), comparable to the expression induced by ectopic overexpression (FIG. 6I), together with induction of NE markers and suppression of AR. In the absence of exogenous SOX2 expression, selinexor was able to prevent enzalutamide-induced NE marker expression and AR suppression, but these effects were abrogated by expression of exogenous SOX2 (FIGS. 6I-6J). However, SOX2 ectopic overexpression was not able to revert the increased sensitivity to selinexor reported after inactivation of TP53 and RB1 (FIGS. 2C and 7B). We tested whether overexpression of factors other than SOX2, which of our data implicate in NE transformation, would rescue the NE phenotype inhibited by selinexor in these models. Overexpression of POU3F2, ONECUT2, or a constitutively active isoform of AKT (myrAKT), did not revert selinexor-induced NE marker expression (FIG. 7C), suggesting that SOX2 might regulate NE transformation upstream any of these factors. These results implicate prevention of SOX2 induction as a mechanism by which exportin 1 inhibition interferes with NE transformation.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and to the extent they are not inconsistent with the explicit teachings of this specification.

### REFERENCES

1. Marcoux, N. et al. EGFR-mutant adenocarcinomas that transform to small-cell lung cancer and other neuroendocrine carcinomas: Clinical outcomes. J. Clin. Oncol. 37, 278-285 (2019).
2. Aggarwal, R. et al. Clinical and genomic characterization of treatment-emergent small-cell neuroendocrine prostate cancer: A multi-institutional prospective study. J. Clin. Oncol. 36, 2492-2503 (2018).
3. Rudin, C. M., Brambilla, E., Faivre-Finn, C. & Sage, J. Small-cell lung cancer. Nat. Rev. Dis. Prim. 7, (2021).
4. Offin, M. et al. Concurrent RB1 and TP53 Alterations Define a Subset of EGFR-Mutant Lung Cancers at risk for Histologic Transformation and Inferior Clinical Outcomes. J. Thorac. Oncol. 14, 1784-1793 (2019).
5. Quintanal-Villalonga, Á. et al. Lineage plasticity in cancer: a shared pathway of therapeutic resistance. Nat. Rev. Clin. Oncol. 17, 360-371 (2020).
6. Quintanal-Villalonga, A. et al. Multi-omic analysis of lung tumors defines pathways activated in neuroendocrine transformation. Cancer Discov. candisc.1863.2021 (2021) doi: 10.1158/2159-8290.cd-20-1863.
7. Mu, P. et al. SOX2 promotes lineage plasticity and antiandrogen resistance in TP53- and RB1-deficient prostate cancer. Science (80-.). 355, 84-88 (2017).
8. Zhang, Y. et al. Androgen deprivation promotes neuroendocrine differentiation and angiogenesis through CREB-EZH2-TSP1 pathway in prostate cancers. Nat. Commun. 9, (2018).
9. Quintanal-Villalonga, A. et al. Comprehensive molecular characterization of lung tumors implicates AKT and MYC signaling in adenocarcinoma to squamous cell transdifferentiation. J. Hematol. Oncol. 14, 1-19 (2021).
10. Niederst, M. J. et al. RB loss in resistant EGFR mutant lung adenocarcinomas that transform to small-cell lung cancer. Nat. Commun. 6, 199-203 (2015).
11. Quintanal-Villalong, A. et al. Inhibition of XPO1 Sensitizes Small Cell Lung Cancer to First- and Second-Line Chemotherapy. Cancer Res. 82, 472-483 (2022).
12. Wang, A. Y. & Liu, H. The past, present, and future of CRM1/XPO1 inhibitors. Stem Cell Investig. 6, (2019).
13. Nachmias, B. & Schimmer, A. D. Targeting nuclear import and export in hematological malignancies. Leukemia 34, 2875-2886 (2020).
14. Tzelepi, V. et al. Modeling a lethal prostate cancer variant with small-cell carcinoma features. Clin. Cancer Res. 18, 666-677 (2012).
15. Abida, W. et al. Genomic correlates of clinical outcome in advanced prostate cancer. Proc. Natl. Acad. Sci. U.S.A. 166, 11428-11436 (2019).
16. Park, C. K., Oh, I. J. & Kim, Y. C. Is transformed small cell lung cancer (SCLC) different from de novo SCLC? Transl. Cancer Res. 8, 346-349 (2019).
17. True, L. D. et al. A Neuroendocrine/Small Cell Prostate Carcinoma Xenograft-LuCaP 49. Am J Pathol. 161, 705-715 (2002).
18. Park, J. W. et al. Reprogramming normal human epithelial tissues to a common, lethal neuroendocrine cancer lineage. Science (80-.). 362, 91-95 (2018).
19. Fares, A. F. et al. ALK-rearranged lung adenocarcinoma transformation into high-grade large cell neuroendocrine carcinoma: Clinical and molecular description of two cases. Lung Cancer 146, 350-354 (2020).
20. Dardenne, E. et al. N-Myc Induces an EZH2-Mediated Transcriptional Program Driving Neuroendocrine Prostate Cancer. Cancer Cell 30, 563-577 (2016).
21. Sheng Yu Ku, Spencer Rosario, Yanqing Wang, Ping Mu, Mukund Seshadri, Zachary W. Goodrich, Maxwell M. Goodrich, David P. Labbé, Eduardo Cortes Gomez, Jianmin Wang, Henry W. Long, Bo Xu, Myles Brown, Massimo Loda, Charles L. Sawyers, Leigh Ellis, and D. W. G. Rb1 and Trp53 Cooperate to Suppress Prostate Cancer Lineage Plasticity, Metastasis, and Antiandrogen Resistance. Science (80-.). 355, 78-83 (2017).
22. Quintanal-Villalonga, Á. et al. FGFR1 and FGFR4 oncogenicity depends on n-cadherin and their co-expression may predict FGFR-targeted therapy efficacy. EBioMedicine 53, 1-15 (2020).
23. Ianevski, A., Giri, A. K. & Aittokallio, T. SynergyFinder 2.0: Visual analytics of multi-drug combination synergies. Nucleic Acids Res. 48, W488-W493 (2021).
24. Gardner, E. E. et al. Chemosensitive Relapse in Small Cell Lung Cancer Proceeds through an EZH2-SLFN11 Axis. Cancer Cell 31, 286-299 (2017).
25. Patro, R., Duggal, G., Love, M. I., Irizarry, R. A. & Kingsford, C. Salmon provides fast and bias-aware quantification of transcript expression. Nat. Methods 14, 417-419 (2017).
26. Zerbino, D. R. et al. Ensembl 2018. Nucleic Acids Res. 46, D754-D761 (2018).
27. Pimentel, H., Bray, N. L., Puente, S., Melsted, P. & Pachter, L. Differential analysis of RNA-seq incorporating quantification uncertainty. Nat. Methods 14, 687-690 (2017).
28. Chen, J. et al. Genomic landscape of lung adenocarcinoma in East Asians. Nat. Genet. 52, 177-186 (2020).
29. Abeshouse, A. et al. The Molecular Taxonomy of Primary Prostate Cancer. Cell 163, 1011-1025 (2015).
30. Ritchie, M. E. et al. Limma powers differential expression analyses for RNA-sequencing and microarray studies. Nucleic Acids Res. 43, e47 (2015).
31. Subramanian, A. et al. Gene set enrichment analysis: A knowledge-based approach for interpreting genome-wide expression profiles. Proc. Natl. Acad. Sci. U.S.A. 102, 15545-15550 (2005).
32. Liberzon, A. et al. Molecular signatures database (MSigDB) 3.0. Bioinformatics 27, 1739-1740 (2011).
33. Yu, G., Wang, L. G., Han, Y. & He, Q. Y. ClusterProfiler: An R package for comparing biological themes among gene clusters. Omi. A J. Integr. Biol. 16, 284-287 (2012).
34. Yi, L., Pimentel, H., Bray, N. L. & Pachter, L. Gene-level differential analysis at transcript-level resolution. Genome Biol. 19, 1-11 (2018).
35. Langmead, B. & Salzberg, S. L. Fast gapped-read alignment with Bowtie 2. Nat.Methods 9, 357-359 (2012).
36. Zhang, Y. et al. Model-based analysis of ChIP-Seq (MACS). Genome Biol. 9, (2008).
37. Liao, Y., Smyth, G. K. & Shi, W. FeatureCounts: An efficient general purpose program for assigning sequence reads to genomic features. Bioinformatics 30, 923-930 (2014).
38. Love, M. I., Huber, W. & Anders, S. Moderated estimation of fold change and dispersion for RNA-seq data with DESeq2. Genome Biol. 15, 1-21 (2014).
39. Lopez-Delisle, L. et al. pyGenomeTracks: reproducible plots for multivariate genomic datasets. Bioinformatics 37, 422-423 (2021).

1. A method for treating or preventing neuroendocrine tumor formation in a patient diagnosed with adenocarcinoma comprising administering to the patient (a) an effective amount of an XPO1 inhibitor or (b) an effective amount of an XPO1 inhibitor and an effective amount of at least one chemotherapeutic drug, wherein the adenocarcinoma exhibits reduced expression and/or activity of TP53 and RB1.

2. (canceled)

3. The method of claim 1, wherein the adenocarcinoma comprises (a) a genetic mutation, an indel, a copy number alteration or epigenetic downregulation in TP53 and (b) a genetic mutation, an indel, a copy number alteration or epigenetic downregulation in RB1.

4. The method of claim 1, wherein the XPO1 inhibitor and the at least one chemotherapeutic drug are administered sequentially, simultaneously, or separately.

5. The method of claim 1, wherein the at least one chemotherapeutic drug is administered orally, intranasally, parenterally, intravenously, intramuscularly, intraperitoneally, or subcutaneously, intratumorally, or topically.

6. The method of claim 1, wherein the at least one chemotherapeutic drug is an alkylating agent, a platinum agent, a taxane, a vinca agent, an aromatase inhibitor, a cytostatic alkaloid, a cytotoxic antibiotic, an antimetabolite, an endocrine/hormonal agent, or a bisphosphonate therapy agent.

7. The method of claim 1, wherein the at least one chemotherapeutic drug comprises one or more agents selected from the group consisting of cyclophosphamide, fluorouracil (or 5-fluorouracil or 5-FU), methotrexate, edatrexate (10-ethyl-10-deaza-aminopterin), thiotepa, carboplatin, cisplatin, taxanes, paclitaxel, protein-bound paclitaxel, docetaxel, vinorelbine, tamoxifen, raloxifene, toremifene, fulvestrant, gemcitabine, irinotecan, ixabepilone, temozolmide, topotecan, vincristine, vinblastine, eribulin, mutamycin, capecitabine, anastrozole, exemestane, letrozole, risedronate, pamidronate, ibandronate, alendronate, denosumab, zoledronate, tykerb, anthracyclines (e.g., daunorubicin and doxorubicin), oxaliplatin, melphalan, etoposide, mechlorethamine, bleomycin, microtubule poisons, annonaceous acetogenins, abraxane, leucovorin, nab-paclitaxel, everolimus, pegylated-hyaluronidase, pemetrexed, folinic acid, MK2206, GDC-0449, IPI-926, M402, LY293111 or combinations thereof.

8. A method for preventing neuroendocrine tumor formation in a patient diagnosed with adenocarcinoma comprising administering to the patient an effective amount of an XPO1 inhibitor and an effective amount of an androgen receptor (AR) inhibitor, optionally wherein the adenocarcinoma exhibits reduced expression and/or activity of TP53 and RB1.

9. The method of claim 8, wherein the XPO1 inhibitor and the AR inhibitor are administered sequentially, simultaneously, or separately.

10. The method of claim 8, wherein the AR inhibitor is administered orally, intranasally, parenterally, intravenously, intramuscularly, intraperitoneally, or subcutaneously, intratumorally, or topically.

11. The method of claim 8, wherein the AR inhibitor comprises one or more agents selected from the group consisting of apalutamide, bicalutamide, darolutamide, enzalutamide, flutamide, abiraterone acetate, ARN-509, and nilutamide.

12. A method for preventing neuroendocrine tumor formation in a patient diagnosed with adenocarcinoma comprising administering to the patient an effective amount of an XPO1 inhibitor and an effective amount of an epidermal growth factor receptor (EGFR) inhibitor (EGFRi), optionally wherein the adenocarcinoma exhibits reduced expression and/or activity of TP53 and RB1.

13. The method of claim 12, wherein the XPO1 inhibitor and the EGFRi are administered sequentially, simultaneously, or separately.

14. The method of claim 12, wherein the EGFRi is administered orally, intranasally, parenterally, intravenously, intramuscularly, intraperitoneally, or subcutaneously, intratumorally, or topically.

15. The method of claim 12, wherein the EGFRi comprises one or more agents selected from the group consisting of osimertinib, afatinib, erlotinib, gefitinib, icotinib, dacomitinib, rociletinib, olmutinib, cetuximab, panitumumab, nimotuzumab, and necitumumab.

16. The method of claim 8, wherein the adenocarcinoma comprises (a) a genetic mutation, an indel, a copy number alteration or epigenetic downregulation in TP53 and (b) a genetic mutation, an indel, a copy number alteration or epigenetic downregulation in RB1.

17. The method of claim 1, wherein the patient is diagnosed with TP53^{-/-} and RB1^{-/-} mutant adenocarcinoma, optionally wherein the TP53^{-/-} and RB1^{-/-} mutant adenocarcinoma is lung adenocarcinoma or prostate adenocarcinoma.

18. (canceled)

19. The method of claim 1, wherein the XPO1 inhibitor comprises one or more agents selected from the group consisting of KPT-330 (selinexor), KOS-2462, KPT-335 (verdinexor), KPT-176, KPT-127, KPT-185, KPT-276,KPT-251, KPT-205, KPT-227, Leptomycin B (LMB), ratjadone, goniothalamin, N-azolylacrylates, anguinomycin, and CBS9106.

20. The method of claim 1, wherein the patient is non-responsive to at least one prior line of cancer therapy.

21. The method of claim 20, wherein the at least one prior line of cancer therapy is chemotherapy.

22. The method of claim 1, wherein the XPO1 inhibitor is administered orally, intranasally, parenterally, intravenously, intramuscularly, intraperitoneally, or subcutaneously, intratumorally, or topically.
